(19) United States
(12) Patent Application Publication
(10) Pub. No.: US 2006/0199250 A1
(43) Pub. Date: Sep. 7, 2006

(54) Method for generating a mutant protein which efficiently binds a target molecule

(76) Inventors: Huimin Zhao, Champaign, IL (US); Karuppiah Chockalingam, Champaign, IL (US)

Correspondence Address: Licata & Tyrrell P.C., 66 E. Main Street, Marlton, NJ, 08053, US

(21) Appl. No.: 11/368,891

(22) Filed: Mar. 6, 2006

Related U.S. Application Data
(60) Provisional application No. 60/658,986, filed on Mar. 4, 2005.

(51) Int. Cl. C07K 14/47; C12P 21/06

(52) U.S. Cl. 435/69.1; 435/320.1; 435/325; 435/455; 530/350

(57) ABSTRACT

The present invention relates to a method for generating a mutant protein which efficiently binds a target molecule. The method of the invention employs saturation mutagenesis and random mutagenesis approaches producing one or more mutant proteins with enhanced binding efficiency for a target molecule compared to binding of a wild-type protein to the target molecule. Mutant proteins generated in accordance with the present invention are also provided.

### INTRODUCTION

This application claims benefit of U.S. Provisional Patent Application Ser. No. 60/658,986, filed Mar. 4, 2005, the contents of which is incorporated herein by reference in its entirety.

This invention was made with government support under Grant Number BES-0348107, awarded by The National Science Foundation. The Government may have certain rights to this invention.

### BACKGROUND OF THE INVENTION

The ability to manipulate naturally occurring proteins to bind and respond to synthetic ligands in a manner independent, or orthogonal, from the influence of natural proteins and ligands, constitutes an important aspect of protein engineering (Koh (2002) Chem. Biol. 9:17-23). Such a tool has important utility in the creation of gene switches for the control of heterologous gene expression in applications such as gene therapy and metabolic engineering, as well as in the selective regulation of cellular processes such as apoptosis, genetic recombination, signal transduction, and motor protein function (Harvey & Caskey (1998) Curr. Opin. Chem. Biol. 2:512-518; Fussenegger (2001) Biotechnol. Progr. 17:1-51; Bishop, et al. (2000) Annu. Rev. Bioph. Biom. 29:577-606).

A number of synthetic ligand-mutant receptor pairs have been created that are orthogonal to the analogous natural interaction to varying degrees. Amongst the proteins described, nuclear hormone receptors are commonly used due to their “gene switch-like” attributes, rapid induction kinetics, dose-dependent ligand response, and readily interchangeable functional modules (Nagy & Schwabe (2004) Trends Biochem. Sci. 29:317-324; Rich, et al. (2002) Proc. Natl. Acad. Sci. USA 99:8562-8567; Braselmann, et al. (1993) Proc. Natl. Acad. Sci. USA 90:1657-1661; Wang, et al. (1997) Nat. Biotechnol. 15:239-243; Yaghmai & Cutting (2002) Mol. Ther. 5:685-694; Ansari & Mapp (2002) Curr. Opin. Chem. Biol. 6:765-772). Although a number of methods have been used to engineer novel and specific receptor-ligand pairs from nuclear hormone receptors, there remains a need to develop a simple, generally applicable protein engineering approach. The present invention meets this need in the art.

### SUMMARY OF THE INVENTION

The present invention is a method for generating a mutant protein which efficiently binds a target molecule. The method involves the steps of identifying one or more amino acid residues of a binding site of a wild-type protein for a target molecule; subjecting at least one amino acid residue of the binding site to saturation mutagenesis; selecting for at least one binding site mutant protein with enhanced binding efficiency for the target molecule compared to binding efficiency of the wild-type protein for the target molecule; subjecting the binding site mutant protein to random mutagenesis; and selecting for at least one mutant protein with enhanced binding efficiency for the target molecule compared to binding efficiency of the wild-type protein or binding site mutant protein for the target molecule thereby generating a mutant protein which efficiently binds the target molecule. Mutant proteins generated in accordance with the method of the present invention are also provided.

### BRIEF DESCRIPTION OF THE DRAWINGS

FIG. 1 depicts an exemplary method for generating orthogonal receptor-ligand pairs.

FIG. 2 depicts an exemplary selection of amino acid residues of the binding site of human estrogen receptor α (hERα) for mutagenesis.

FIGS. 3A-3B depict the transactivation profiles in yeast two-hybrid cells for wild-type hERα and 4,4′-dihydroxybenzil (DHB) mutant proteins in response to DHB (FIG. 3A) and 17β-estradiol (E₂).

FIG. 4 depicts the transactivation profiles in HEC-1 cells for wild-type hERα and DHB mutant proteins.

FIGS. 5A-5B depict yeast dose response curves for 2,4-di(4-hydroxyphenyl)-5-ethylthiazole (L9; FIG. 5A) and 17β-estradiol (E₂; FIG. 5B)) of the L9-selective receptor mutants generated by either saturation mutagenesis of ligand binding pocket sites (H14, U5, N5, Y3, K10) or error-prone PCR of the hERα ligand binding domain (X10).

### DETAILED DESCRIPTION OF THE INVENTION

The present invention relates to methods and compositions for the generation of mutant proteins with significantly altered selectivity or binding efficiency for a target molecule as compared to the binding efficiency of wild-type protein for the target molecule. By “target molecule” herein is meant any molecule for which an interaction is sought. Target molecules that are capable of binding to a protein and/or being acted upon by a protein are used in the methods and compositions described herein. Suitable target molecules include, but are not limited to, ligands, enzyme substrates, and chemical moieties, such as small molecules, drugs, and ions.

In accordance with the present invention, a wild-type protein whose selectivity or binding efficiency for a target molecule is to be altered can be any protein with a binding site for which a cognate molecule is known in the art to bind. As used herein, cognate is used in the conventional sense to refer to two biomolecules that typically interact (e.g., a receptor and its ligand). In general, the target molecule and a cognate molecule can share common structural features; however, the wild-type protein does not bind or binds with a low efficiency to the target molecule. By mutating the wild-type protein, efficiency of binding to the target molecule is enhanced. Examples of suitable protein-target molecule pairs include, but are not limited to, receptor-ligand pairs, enzyme-substrate pairs, antibody-antigen pairs, etc.

The library strategies described herein contain stepwise site-saturation mutagenesis of individual residues identified by a structure-based design method as contacting target molecules. Each mutagenic library step is generally accompanied by a phenotypic screen for a mutant receptor(s) with enhanced target molecule selectivity or binding, followed by random point mutagenesis and phenotypic screening for further binding efficiency-enhanced mutants.

The stepwise, individual, site-saturation mutagenesis/random point mutagenesis strategy described herein differs from other approaches that have been used for creating novel protein-target molecule pairs. In particular, the current library creation strategy can be generalized to a number of protein-target molecule systems, provided sufficient structural information about the protein is available, without having to choose specific allowable amino acid substitutions for randomized target molecule-contacting sites on the protein.

Further, as there are only 32 possible codon substitutions, or 19 possible amino acid substitutions per site for the instant saturation mutagenesis libraries, subjecting 96 transformants to screening in a convenient 96-well plate format is sufficient to represent most, if not all, the possible library variants. In contrast, conventional combinatorial randomization strategies rely on the dominant presence of selective variants within a large library (Schwimmer, et al. (2004) Proc. Natl. Acad. Sci. USA 101:14707-14712); despite the ˜3×10⁶ possible codon combinations, only ˜3.8×10⁵ transformants were subjected to selection.

Moreover, the instant library size is very small for saturation mutagenesis, wherein essentially all randomized variants are subjected to simultaneous positive screening and negative screening. Advantageously, the instant stepwise site saturation mutagenesis strategy allows every site in a binding site or binding domain to randomize to all 20 possible amino acids. In contrast, methods for creating a library of protein variants based on single base pair substitutions at the DNA level can access only a limited number (˜6 on average) of amino acid substitutions per residue to identify variants with significantly altered target molecule selectivity using an error-prone PCR-based random mutagenesis strategy (see, e.g., Miller & Whelan (1998) J. Steroid Biochem. 64:129-135; Whelan & Miller (1996) J. Steroid Biochem. 58:3-12).

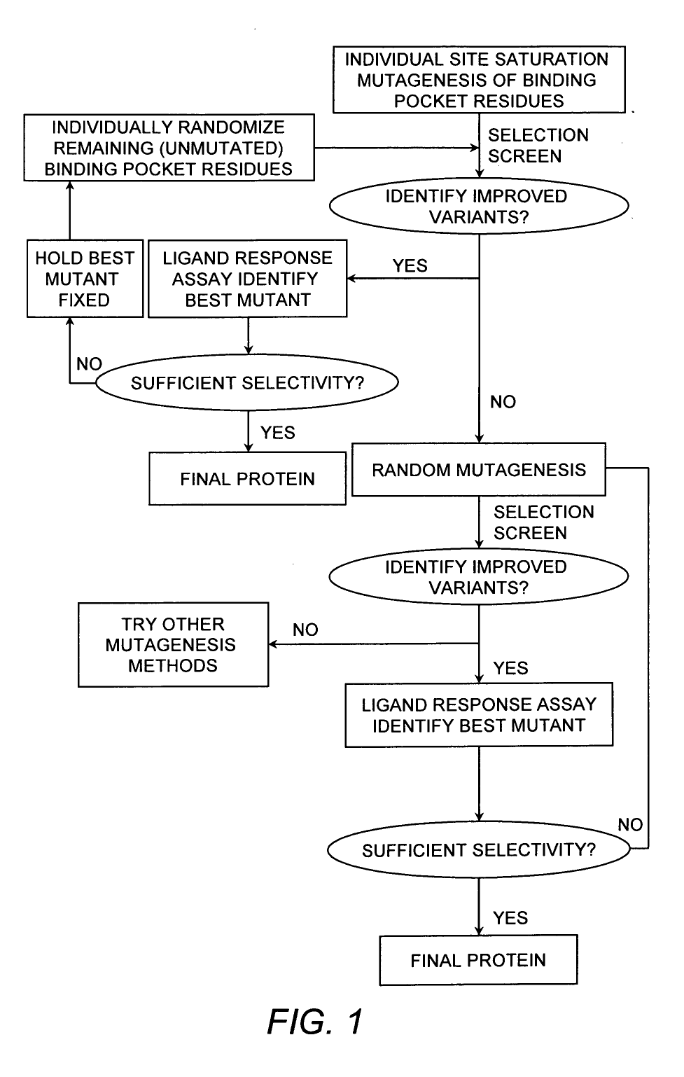

FIG. 1 depicts an exemplary embodiment for creating libraries for generating novel protein-target molecule pairs. Typically, all of the amino acid residues in a protein that are involved in binding a target molecule are identified prior to the application of a stepwise targeted saturation mutagenesis procedure. For example, a molecular docking program is used to identify key amino acid residues involved in the binding of a target molecule to the protein. Subsequently, a stepwise saturation mutagenesis procedure is independently applied to each of the amino acid residues identified as being involved in the binding of a target molecule, or to a subset of the amino acids identified as being involved in the binding of a target molecule. The resulting library is screened and mutant(s) that exhibit the greatest increase in binding or activation in response to the target molecule as compared to the wild-type protein are selected. One, two, three, four, or more rounds of individual targeted saturation mutagenesis can be applied to the remaining unmutated amino acid residues involved in binding the target molecule until no further increase in binding or activation in response to the target molecule is observed.

In some embodiments, random mutagenesis is performed on some or all of the amino acid residues of the mutant protein(s) identified from the saturation mutagenesis libraries as exhibiting the greatest increase in binding or activation in response to the target molecule as compared to the wild-type protein. Generally, random mutagenesis is used to generate mutants of mutations outside of the target molecule binding domain, but which affect target molecule selectively. One or more rounds of random mutagenesis can be performed until at least one mutant protein with the desired level of activity toward the target molecule is obtained.

The number of saturation mutagenesis and random mutagenesis libraries employed in the methods described herein is not critical, and depends in part, on obtaining at least one mutant protein with the desired level of activity toward the target molecule. Generally, one or more saturation mutagenesis libraries and one or more random mutagenesis libraries are generated using the methods described herein. For example, in some embodiments, a first saturation mutagenesis library and a second random mutagenesis library are generated. In other embodiments, two or more saturation mutagenesis libraries, and one or more random mutagenesis libraries are generated. In other embodiments, three, four, or more saturation mutagenesis libraries, and one or more random mutagenesis libraries are generated.

In the present method, the primary goal is to create a mutant protein which efficiency binds to a target molecule, wherein binding efficiency will depend on the nature of the protein and/or target molecule. For example, in the case of a wild-type protein which exhibits no binding affinity for a target molecule, any increase in binding of a mutant protein to the target molecule as compared to the wild-type protein is considered efficient binding of the target molecule to the mutant protein. Moreover, in the case of a wild-type protein which exhibits minimal binding affinity for a target molecule, a two-fold or greater increase in binding of the mutant protein to the target molecule as compared to the wild-type protein is considered efficient binding of the target molecule to the mutant protein. Typically, the level of activation or efficiency of binding between the mutant protein and the target molecule increases with an increase in mutagenesis steps so that the target molecule efficiently binds to the mutant protein. In contrast, the level of activation or efficiency of binding between the mutant protein and the native cognate molecule decreases. For example, the mutant protein generated at the first targeted saturation mutagenesis step can exhibit a binding efficiency between 10-fold to 100-fold greater than the wild-type protein, and exhibit a binding efficiency toward the native cognate molecule that is decreased between 1-fold to 100-fold as compared to the wild-type protein. Subsequent rounds of library generation can generate mutant proteins with binding efficiencies for the target molecule between 10-fold to 10³-fold greater than the wild-type protein and exhibit binding efficiencies toward the native cognate molecule that is decreased between 10²-fold to 10¹⁰-fold as compared to the wild-type protein. Generally, binding efficiency is defined by the level of activation (e.g., the EC₅₀ for a receptor and ligand), enzymatic activity, selectively, binding affinity (e.g., equilibrium constant of an antibody-antigen interaction), or as an efficacy measurement.

In some embodiments, binding efficiency of a mutant protein and a target molecule is expressed as an EC₅₀ value in nM. As will be appreciated by a person skilled in the art, the range of EC₅₀ values observed depends in part on the assay system. Typically, higher EC₅₀ values are observed in yeast cells than in mammalian cells. For example, in some embodiments, depending on the cells used in the assay, EC₅₀ values range from 0.1 nM to 1000 nM. In other embodiments, the EC₅₀ values range from 0.1 nM to 500 nM. In yet other embodiments, EC₅₀ values range from 0.1 nM to 100 nM.

Alternatively, the binding efficiency of a mutant protein and the target molecule is expressed as an efficacy measurement. Efficacy, given as a fold-increase in activation, is defined as the maximum increase in activation of the mutant protein relative to the activation of the wild-type protein with a given concentration of a target molecule. For example, in some embodiments, the efficacy of a mutant protein is from 2-fold to 10¹⁰-fold for the target molecule. In other embodiments, the efficacy of a mutant protein is at least 10²-fold, 10³-fold, 10⁴-fold, 10⁵-fold, 10⁶-fold, 10⁷-fold, 10⁸-fold, 10⁹-fold, or 10¹⁰-fold.

In other embodiments, the selectivity of the mutant protein toward the target molecule is measured. Selectivity toward the target molecule is determined by dividing the EC₅₀ of the cognate molecule by the EC₅₀ of the target molecule. For example, in some embodiments, the selectivity of a mutant protein toward the target molecule is from 2 to ≧10⁸. In other embodiments, the selectivity of a mutant protein is at least 10, 100, 1000, or ≧10⁴ for the target molecule.

The binding efficiency or level of activation of the mutant protein(s) by the target molecule is generally selected by the user, depending, in part, on the particular application. For example, in some embodiments, orthogonal receptor-ligand pairs are generated using the methods described herein. By “orthogonal” herein is meant that receptor cannot be activated by endogenous native cognate molecules, and the ligand cannot activate endogenous receptors. Thus, a mutant receptor that is activated only by the target ligand and not by endogenous cognate molecules, as well as a target ligand that activates only a mutant receptor and not endogenous receptors can be achieved. Alternatively, mutant proteins can be generated that exhibit different levels of binding in response to the target molecule and the wild-type cognate molecule. Virtually any existing receptor can be used as the starting point for the generation of an orthogonal receptor-ligand pair.

Any structure-based method for identifying amino acid residues in the protein which contact the target molecule can be used in the methods and compositions described herein. For example, structure can be determined based on sequence identity with a protein having a known three dimensional structure. A number of different programs can be used to identify whether a protein or nucleic acid has sequence identity or similarity to a known sequence. Sequence identity and/or similarity is determined using standard techniques known in the art, including, but not limited to, the local sequence identity algorithm of Smith & Waterman ((1981) Adv. Appl. Math. 2:482), by the sequence identity alignment algorithm of Needleman & Wunsch ((1970) J. Mol. Biol. 48:443), by the search for similarity method of Pearson & Lipman ((1988) Proc. Natl. Acad. Sci. USA 85:2444), by computerized implementations of these algorithms (e.g., GAP, BESTFIT, FASTA, and TFASTA in the Wisconsin Genetics Software Package, Genetics Computer Group, Madison, Wis.), the Best Fit sequence program described by Devereux, et al. ((1984) Nucl. Acid Res. 12:387-395), using the default settings, or by inspection. Percent identity can be calculated by FastDB using the following parameters: mismatch penalty of 1; gap penalty of 1; gap size penalty of 0.33; and joining penalty of 30 (see. e.g., “Current Methods in Sequence Comparison and Analysis,” Macromolecule Sequencing and Synthesis, Selected Methods and Applications, pp 127-149 (1988), Alan R. Liss, Inc.).

Other examples of useful algorithms include, but are not limited to, PILEUP, which uses a simplification of the progressive alignment method of Feng & Doolittle ((1987) J. Mol. Evol. 35:351-360), which is similar to that described by Higgins & Sharp ((1989) CABIOS 5:151-153); the BLAST algorithm (see, e.g., Altschul, et al. (1990) J. Mol. Biol. 215:403-410; Altschul, et al. (1997) Nucleic Acids Res. 25:3389-3402; Karlin, et al. (1993) Proc. Natl. Acad. Sci. USA 90:5873-5787); WU-BLAST-2 program which was obtained from Altschul, et al. ((1996) Meth. Enzymol. 266:460-480); and gapped BLAST as reported by Altschul, et al. ((1997) Nucl. Acids Res. 25:3389-3402).

In some embodiments, models of the wild-type protein complexed with the target molecule are built using the Molecular Operating Environment (MOE) software (Chemical Computing Group, Montreal, Canada). Examples of other suitable modeling programs include, but are not limited to, structure-based alignment programs. See, for example, Doyle, et al. (2001) J. Am. Chem. Soc. 123:11367-11373; Schwimmer, et al. (2004) Proc. Natl. Acad. Sci. USA 101:14707-14712.

In some embodiments, the choice of which amino acid residue(s) to mutate is determined by examining the X-ray crystal structure of related protein(s) complexed with a molecule having a structure similar to the target molecule.

In particular embodiments, all of the amino acid residues that are capable of contacting the target molecule are mutated using any one of the site-directed saturation mutagenesis techniques described herein. In other embodiments, some or a subset of the amino acid residues that are capable of contacting the target molecule are mutated, and the remaining amino acid residues are fixed. Amino acid residues that can be fixed include, but are not limited to, residues that confer desired protein properties, such as structural or biological functional properties. For example, residues which are known to be important for biological activity, such as residues which form the active site of an enzyme, the substrate binding site of an enzyme, the binding site for a binding partner (ligand/receptor, antigen/antibody, etc.), phosphorylation or glycosylation sites, or structurally important residues, such as cysteine residues that participate in disulfide bridges, metal binding sites, critical hydrogen bonding residues, residues critical for backbone conformation such as proline or glycine, residues critical for packing interactions, etc. can be fixed.

In some embodiments, fixed residues that confer desired protein properties are specifically targeted for site-directed saturation mutagenesis. For example, this strategy can be used to alter properties such as binding affinity, binding specificity and catalytic efficiency. A region such as a binding site or active site can be defined, for example, to include all residues within a certain distance, for example 4-10 Å, or preferably 5 Å, of the residues that are in van der Waals contact with the substrate or ligand. Alternatively, a region such as a binding site or active site can be defined using experimental results, for example, a binding site could include all positions at which mutation has been shown to affect binding.

In certain embodiments, some amino acid residues in the protein which contact the target molecule are held constant, or are selected from a limited number of possibilities. For example, in some embodiments, the nucleotides or amino acid residues are randomized within a defined class, for example, by hydrophobic amino acid residues hydrophilic amino acid residues, acidic amino acid residues, basic amino acid residues, polar amino acid residues.

As used in the context of the present invention, “hydrophilic amino acid or residue” refers to an amino acid or residue having a side chain exhibiting a hydrophobicity of less than zero according to the normalized consensus hydrophobicity scale of Eisenberg, et al. ((1984) J. Mol. Biol. 179:125-142). Genetically encoded hydrophilic amino acids include L-Thr (T), L-Ser (S), L-His (H), L-Glu (E), L-Asn (N), L-Gln (Q), L-Asp (D), L-Lys (K) and L-Arg (R).

“Acidic amino acid or residue” refers to a hydrophilic amino acid or residue having a side chain exhibiting a pK value of less than about 6 when the amino acid is included in a peptide or polypeptide. Acidic amino acids typically have negatively charged side chains at physiological pH due to loss of a hydrogen ion. Genetically encoded acidic amino acids include L-Glu (E) and L-Asp (D).

“Basic amino acid or residue” refers to a hydrophilic amino acid or residue having a side chain exhibiting a pK value of greater than about 6 when the amino acid is included in a peptide or polypeptide. Basic amino acids typically have positively charged side chains at physiological pH due to association with hydronium ion. Genetically encoded basic amino acids include L-His (H), L-Arg (R) and L-Lys (K).

“Polar amino acid or residue” refers to a hydrophilic amino acid or residue having a side chain that is uncharged at physiological pH, but which has at least one bond in which the pair of electrons shared in common by two atoms is held more closely by one of the atoms. Genetically encoded polar amino acids include L-Asn (N), L-Gln (Q), L-Ser (S) and L-Thr (T).

“Hydrophobic amino acid or residue” refers to an amino acid or residue having a side chain exhibiting a hydrophobicity of greater than zero according to the normalized consensus hydrophobicity scale of Eisenberg, et al. ((1984) supra). Genetically encoded hydrophobic amino acids include L-Pro (P), L-Ile (I), L-Phe (F), L-Val (V), L-Leu (L), L-Trp (W), L-Met (M), L-Ala (A) and L-Tyr (Y).

“Aromatic amino acid or residue” refers to a hydrophilic or hydrophobic amino acid or residue having a side chain that includes at least one aromatic or heteroaromatic ring. The aromatic or heteroaromatic ring may contain one or more substituents such as —OH, —OR″, —SH, —SR″, —CN, halogen (e.g., —F, —Cl, —Br, —I), —NO₂, —NO, —NH₂, —NHR″, —NR″R″, —C(O)R″, —C(O)O^{—}, —C(O)OH, —C(O)OR″, —C(O)NH₂, —C(O)NHR″, —C(O)NR″R″ and the like, where each R″ is independently (C₁-C₆) alkyl, substituted (C₁-C₆) alkyl, (C₂-C₆) alkenyl, substituted (C₂-C₆) alkenyl, (C₂-C₆) alkynyl, substituted (C₂-C₆) alkynyl, (C₅-C₁₀) aryl, substituted (C₅-C₁₀) aryl, (C₆-C₁₆) arylalkyl, substituted (C₆-C₁₆) arylalkyl, 5-10 membered heteroaryl, substituted 5-10 membered heteroaryl, 6-16 membered heteroarylalkyl or substituted 6-16 membered heteroarylalkyl. Genetically encoded aromatic amino acids include L-Phe (F), L-Tyr (Y) and L-Trp (W). Although owing to the pKa of its heteroaromatic nitrogen atom L-His (H) is classified as a basic residue, as its side chain includes a heteroaromatic ring, it can also be classified as an aromatic residue.

“Non-polar amino acid or residue” refers to a hydrophobic amino acid or residue having a side chain that is uncharged at physiological pH and which has bonds in which the pair of electrons shared in common by two atoms is generally held equally by each of the two atoms (i.e., the side chain is not polar). Genetically encoded non-polar amino acids include L-Leu (L), L-Val (V), L-Ile (I), L-Met (M) and L-Ala (A).

“Aliphatic Amino Acid or Residue” refers to a hydrophobic amino acid or residue having an aliphatic hydrocarbon side chain. Genetically encoded aliphatic amino acids include L-Ala (A), L-Val (V), L-Leu (L) and L-Ile (I).

“Small amino acid or residue” refers to an amino acid or residue having a side chain that is composed of a total of three or fewer carbon and/or heteroatoms (excluding the α-carbon and hydrogens). The small amino acids or residues can be further categorized as aliphatic, non-polar, polar or acidic small amino acids or residues, in accordance with the above definitions. Genetically-encoded small amino acids include Gly, L-Ala (A), L-Val (V), L-Cys (C), L-Asn (N), L-Ser (S), L-Thr (T) and L-Asp (D).

“Hydroxyl-containing residue” refers to an amino acid containing a hydroxyl (—OH) moiety. Genetically-encoded hydroxyl-containing amino acids include L-Ser (S) L-Thr (T) and L-Tyr (Y).

As will be appreciated by those of skill in the art, the above-defined categories are not mutually exclusive. For example, the delineated category of small amino acids includes amino acids from all of the other delineated categories except the aromatic category. Thus, amino acids having side chains exhibiting two or more physico-chemical properties can be included in multiple categories. As a specific example, amino acid side chains having heteroaromatic moieties that include ionizable heteroatoms, such as His, can exhibit both aromatic properties and basic properties, and can therefore be included in both the aromatic and basic categories. The appropriate classification of any amino acid or residue will be apparent to those of skill in the art, especially in light of the detailed disclosure provided herein.

In some embodiments, the amino acid residues in the protein which contact the target molecule are selected from any of the naturally-occurring amino acids. In other embodiments, one or more or synthetic non-encoded amino acids is used to replace one or more of the naturally-occurring amino acid residues. Certain commonly encountered non-encoded amino acids include, but are not limited to: the D-enantiomers of the genetically-encoded amino acids; 2,3-diaminopropionic acid (Dpr); α-aminoisobutyric acid (Aib); ε-aminohexanoic acid (Aha); δ-aminovaleric acid (Ava); N-methylglycine or sarcosine (MeGly or Sar); ornithine (Orn) ; citrulline (Cit); t-butylalanine (Bua); t-butylglycine (Bug); N-methylisoleucine (MeIle); phenylglycine (Phg); cyclohexylalanine (Cha); norleucine (Nle); naphthylalanine (Nal); 2-chlorophenylalanine (Ocf); 3-chlorophenylalanine (Mcf); 4-chlorophenylalanine (Pcf); 2-fluorophenylalanine (Off); 3-fluorophenylalanine (Mff); 4-fluorophenylalanine (Pff); 2-bromophenylalanine (Obf); 3-bromophenylalanine (Mbf); 4-bromophenylalanine (Pbf); 2-methylphenylalanine (Omf); 3-methylphenylalanine (Mmf); 4-methylphenylalanine (Pmf); 2-nitrophenylalanine (Onf); 3-nitrophenylalanine (Mnf); 4-nitrophenylalanine (Pnf); 2-cyanophenylalanine (Ocf); 3-cyanophenylalanine (Mcf); 4-cyanophenylalanine (Pcf); 2-trifluoromethylphenylalanine (Otf); 3-trifluoromethylphenylalanine (Mtf); 4-trifluoromethylphenylalanine (Ptf); 4-aminophenylalanine (Paf); 4-iodophenylalanine (Pif); 4-aminomethylphenylalanine (Pamf); 2,4-dichlorophenylalanine (Opef); 3,4-dichlorophenylalanine (Mpcf); 2,4-difluorophenylalanine (Opff); 3,4-difluorophenylalanine (Mpff); pyrid-2-ylalanine (2pAla); pyrid-3-ylalanine (3pAla); pyrid-4-ylalanine (4pAla); naphth-1-ylalanine (1nAla); naphth-2-ylalanine (2nAla); thiazolylalanine (taAla); benzothienylalanine (bAla); thienylalanine (tAla); furylalanine (fAla); homophenylalanine (hPhe); homotyrosine (hTyr); homotryptophan (hTrp); pentafluorophenylalanine (5ff); styrylkalanine (sAla); authrylalanine (aAla); 3,3-diphenylalanine (Dfa); 3-amino-5-phenypentanoic acid (Afp); penicillamine (Pen); 1,2,3,4-tetrahydroisoquinoline-3-carboxylic acid (Tic); β-2-thienylalanine (Thi); methionine sulfoxide (Mso); N(w)-nitroarginine (nArg); homolysine (hLys); phosphonomethylphenylalanine (pmPhe); phosphoserine (pSer); phosphothreonine (pThr); homoaspartic acid (hAsp); homoglutanic acid (hGlu); 1-aminocyclopent-(2 or 3)-ene-4 carboxylic acid; pipecolic acid (PA), azetidine-3-carboxylic acid (ACA); 1-aminocyclopentane-3-carboxylic acid; allylglycine (aOly); propargylglycine (pgGly); homoalanine (hAla); norvaline (nVal); homoleucine (hLeu), homovaline (hVal); homoisolencine (hIle); homoarginine (hArg); N-acetyl lysine (AcLys); 2,4-diaminobutyric acid (Dbu); 2,3-diaminobutyric acid (Dab); N-methylvaline (MeVal); homocysteine (hCys); homoserine (hSer); hydroxyproline (Hyp) and homoproline (hPro). Additional non-encoded amino acids are well-known to those of skill in the art (see, e.g., the various amino acids provided in Fasman (1989) CRC Practical Handbook of Biochemistry and Molecular Biology, CRC Press, Boca Raton, Fla., at pp. 3-70 and the references cited therein). Further, amino acids of the invention can be in either the L- or D-configuration.

Generally, random mutagenesis is performed on all of the amino acid residues of the mutant protein. Thus, the mutant proteins generated using the methods described herein can be composed of anywhere from 0.001% to 99.999% mutated residues out of the total number of residues. For example, mutant proteins of the present invention embrace a change of only a few (or one) residues in the parent or wild-type protein, or most of the residues of the parent or wild-type protein, with all possibilities in between.

Virtually any protein from any source can be used as the parent or starting point for the generation of a novel target molecule/protein pair. The sample containing the protein can be provided from nature or it can be synthesized or supplied from a manufacturing process. For example, the protein can be obtained from an organism, including prokaryotes and eukaryotes, with proteins from bacteria, fungi, viruses, extremophiles such as archaebacteria, insects, fish, mammals, humans, and birds all possible. While the parent or starting point protein is referred to herein as the wild-type protein, the protein does not need to be naturally occurring. For example, the protein could be a designed protein, or a protein selected by a variety of methods including, but not limited to, directed evolution (Farinas, et al. (2001) Curr. Opin. Biotechnol. 12:545-551; Morawski, et al. (2001) Biotechnol. Bioengin. 76:99-107; Stemmer (1994) Nature 370(6488):389-91; Ness, et al. (2000) Adv. Protein. Chem. 55:261-92), DNA shuffling (e.g., technologies available from MAXYGEN®, ENCHIRA, DIVERSA®) or ribosome display (Hanes, et al. (2000) Meth. Enzymol. 328:404-430; Hanes and Pluckthun (1997) Proc. Natl. Acad. Sci. USA 94:4937-4942; Roberts and Szostak (1997) Proc. Natl. Acad. Sci. USA 94:12297-302).

Proteins suitable for use in the methods and compositions described herein, include, but are not limited to, industrial and pharmaceutical proteins, cell surface receptors, antigens, antibodies, cytokines, hormones, transcription factors, signaling modules, cytoskeletal proteins and enzymes. In some embodiments, proteins with known or predictable structures, including mutant proteins, are used. For example, the protein can be any protein for which a three-dimensional structure (i.e., three-dimensional coordinates for each atom of the protein) is known or can be generated. The three-dimensional structures of proteins can be determined using X-ray crystallographic techniques, NMR techniques, de novo modeling, homology modeling, etc. Suitable protein structures include, but are not limited to, all of those found in the Protein Data Base compiled and serviced by the Research Collaboratory for Structural Bioinformatics (RCSB, formerly the Brookhaven National Lab).

Cytokines with known or predictable structures include, e.g., IL-1Ra (+receptor complex), IL-1 (receptor alone), IL-1a, IL-1b including variants and or receptor complex), IL-2, IL-3, L-4, IL-5, IL-6, IL-8, IL-10, IFN-β, INF-γ, IFN-α-2a, FN-α-2B, TNF-α, CD40 ligand (chk), Human Obesity Protein Leptin, Granulocyte Colony-Stimulating Factor, Bone Morphogenetic Protein-7, Ciliary Neurotrophic Factor, Granulocyte-Macrophage Colony-Stimulating Factor, Monocyte Chemoattractant Protein 1, Macrophage Migration Inhibitory Factor, Human Glycosylation-Inhibiting Factor, Human RANTES, Human Macrophage Inflammatory Protein 1 Beta, Human growth hormone, Leukemia Inhibitory Factor, Human Melanoma Growth Stimulatory Activity, neutrophil activating peptide-2, Cc-Chemokine Mcp-3, Platelet Factor M2, Neutrophil Activating Peptide 2, Eotaxin, Stromal Cell-Derived Factor-1, Insulin, Insulin-like Growth Factor I, Insulin-like Growth Factor II, Transforming Growth Factor B1, Transforming Growth Factor B2, Transforming Growth Factor B3, Transforming Growth Factor A, Vascular Endothelial growth factor (VEGF), acidic Fibroblast growth factor, basic Fibroblast growth factor, Endothelial growth factor, Nerve Growth factor, Brain-Derived Neurotrophic Factor, Ciliary Neurotrophic Factor, Platelet Derived Growth Factor, Human Hepatocyte Growth Factor, Fibroblast Growth Factor including but not limited to alternative splice variants, abundant variants, and the like), Glial Cell-Derived Neurotrophic Factor, and hemopoietic receptor cytokines (including but not limited to erythropoietin, thrombopoietin, and prolactin), APM1, and the like.

Extracellular signaling moieties with known or predictable structures include, but are not limited to, sonic hedgehog, protein hormones such as chorionic gonadotrophin and leutenizing hormone.

Transcription factors and other DNA binding proteins of the invention, include but are not limited to, histones, p53, myc, PIT1, NFkB AP1, JUN, KD domain, homeodomain, heat shock transcription factors, stat, zinc finger proteins (e.g., zif268).

Antibodies, antigens, and trojan horse antigens of use as starting proteins, include, but are not limited to, immunoglobulin super family proteins, e.g., CD4 and CD8, Fc receptors, T-cell receptors, MHC-I, MHC-II, CD3, and the like. Immunoglobulin-like proteins are also embraced by the present invention. Such proteins include, e.g., fibronectin, pkd domain, integrin domains, cadherins, invasins, cell surface receptors with Ig-like domains, intrabodies, anti-Her/2 neu antibody (e.g., HERCEPTIN®), anti-VEGF, anti-CD20 (e.g., RITUXAN®), etc.

Receptors embraced by the present invention include, but are not limited to, the extracellular region of human tissue factor cytokine-binding region of Gp130; G-CSF receptor; erythropoietin receptor; fibroblast growth factor receptor; TNF receptor; IL-1 receptor; IL-1 receptor/IL1Ra complex; IL-4 receptor; INF-γ receptor alpha chain; MHC Class I; MHC Class II; T cell receptor; insulin receptor; tyrosine kinase receptors; human growth hormone receptor; G-protein coupled receptors; ABC Transporters/Multidrug resistance proteins such as MRP or MDR1; nuclear hormone receptors such as human estrogen receptor α (SEQ ID NOs:1 and 2; GENBANK Accession No. NM_{—}000125), human estrogen receptor β (SEQ ID NOs:5 and 6; GENBANK Accession No. NM_{—}001437) human progesterone receptor (GENBANK Accession No. NM_{—}000926), human androgen receptor (GENBANK Accession No. NM_{—}000044 or NM_{—}001011645), human glucocorticoid receptor (GENBANK Accession No. NM_{—}000176), human mineralocorticoid receptor (GENBANK Accession No. M16801), human thyroid hormone receptor a (GENBANK Accession No. NM_{—}199334), human thyroid hormone receptor β (GENBANK Accession No. NM_{—}000461); human retinoid receptors such as human retinoid X receptor β (GENBANK Accession No. NM_{—}021976), human retinoid X receptor α (GENBANK Accession No. NM_{—}002957), human retinoic acid receptor α (GENBANK Accession No. NM_{—}000964), human retinoic acid receptor β (GENBANK Accession No. NM_{—}000965 or NM_{—}016152); human vitamin D receptor (GENBANK Accession No. J03258); human peroxisome proliferator-activated receptor α (GENBANK Accession No. Y07619); human peroxisome proliferator-activated receptor γ (GENBANK Accession No. L40904); human peroxisome proliferator-activated receptor (GENBANK Accession No. L02932); liver X receptor; farnesoid X receptor; and ecdysone receptor; aquaporins; transporters; RAGE (receptor for advanced glycan end points); TRK-A; TRK-B; TRK-C; hemopoietic receptors; and the like.

Enzymes with known or predictable structures include, but not limited to, hydrolases such as proteases/proteinases, synthases/synthetases/ligases, decarboxylases/lyases, peroxidases, ATPases, carbohydrases, lipases; isomerases such as racemases, epimerases, tautomerases, or mutases; transferases, hydrolases, kinases, reductases/oxidoreductases, hydrogenases, polymerases, phosphatases, and proteasomes anti-proteasomes, (e.g., MLN341), thioredoxins, homing endonucleases.

Protein domains and motifs are intended to include, but are not limited to, SH-2 domains, SH-3 domains, Pleckstrin homology domains, WW domains, SAM domains, kinase domains, death domains, RING finger domains, Kringle domains, heparin-binding domains, cysteine-rich domains, leucine zipper domains, zinc finger domains, nucleotide binding motifs, transmembrane helices, and helix-turn-helix motifs. Additionally, ATP/GTP-binding site motif A, Ankyrin repeats, fibronectin domain, Frizzled (fz) domain, GTPase binding domain, C-type lectin domain, PDZ domain, Homeobox domain, Krüeppel-associated box (KRAB), cellulose binding domain, leucine zipper, DEAD and DEAH box families, ATP-dependent helicases, HMG1/2 signature, DNA mismatch repair proteins mutL/hexB/PMS1 signature, thioredoxin family active site, annexins repeated domain signature, clathrin light chains signatures, mycotoxin signatures, Staphylococcal enterotoxins/Streptococcal pyrogenic exotoxins signatures, Serpins signature, cysteine proteases inhibitors signature, chaperones, heat shock domains, WD domains, EGF-like domains, immunoglobulin domains, immunoglobulin-like proteins, and the like.

The template nucleic acid for saturation mutagenesis can be a nucleic acid or fragment thereof encoding a wild-type or mutant protein. The template can be used in any of the site-directed saturation mutagenesis techniques described herein to generate a first library of mutant proteins. The first library of mutant proteins is screened, using any one of the screens described herein, to select one or more mutant proteins identified as being capable of binding the target molecule. Mutant proteins which bind the target molecule are isolated, and each of the nucleic acid sequences encoding the proteins are used as templates to generate one or more secondary (i.e., second) libraries of mutant proteins. Depending on the level of binding or activation between the first mutant protein and the target molecule, a secondary library can be generated using either a site-directed saturation mutagenesis technique or any one of the random mutagenesis techniques described herein.

Examples of suitable site-directed saturation mutagenesis techniques include, but are not limited to, “oligonucleotide-directed mutagenesis”, classical site-directed mutagenesis, cassette mutagenesis, and the like. “Oligonucleotide-directed mutagenesis” refers to a process that allows for the generation of site-specific mutations in any cloned DNA segment of interest (see e.g., Ehrlich (1989) PCR Technology, Stockton Press; Oliphant, et al. (1986) Gene 44:177-183; Hermes, et al. (1988) Science 241:53-57; Knowles (1990) Proc. Natl. Acad. Sci. USA 87:696-700), whereas cassette mutagenesis includes the creation of DNA molecules from restriction digestion fragments using nucleic acid ligation, and the random ligation of restriction fragments (see Kikuchi, et al. (1999) Gene 236:159-167). Additionally, cassette mutagenesis can be performed using randomly-cleaved nucleic acids (see Kikuchi, et al. (2000) Gene 243:133-137), by overlap extension PCR as exemplified herein, by PCR-ligation PCR mutagenesis (see, e.g., Ali & Steinkasserer (1995) Biotechniques 18:746-750), by seamless gene engineering using RNA- and DNA-overhang cloning (see Coljee, et al. (2000) Nat. Biotechnol. 18:789-791), by ligation-mediated gene construction, by homologous or non-homologous random recombination (see WO 00/42561 A3; WO 00/42561 A2; WO 00/42560 A3; WO 00/42560 A2; WO 00/42559 A1; WO 00/18906 C2; WO 00/18906 A3; WO 00/18906 A2; and U.S. Pat. Nos. 6,368,861; 6,423,542; 6,376,246; 6,368,861; 6,319,714;), or in vivo using recombination between flanking sequences (see WO 02/10183 A1; Abécassis, et al. (2000) Nucl. Acids Res. 28:e88). Classical site-directed mutagenesis can be carried out using any commercially available kit (e.g., QUICKCHANGE™ available from STRATAGENE®). In addition, regions of the template oligonucleotide encoding the wild-type protein can be mutated in E. coli lacking correct mismatch repair mechanisms (e.g., E. coli XLmutS strain commercially available from STRATAGENE®), or by using phage display techniques to evolve a library (e.g., Long-McGie, et al. (2000) Biotechnol. Bioeng. 68:121-125).

Any one of the random mutagenesis techniques described herein can be used to create libraries of mutant proteins containing one or more mutant proteins which efficiently bind a target molecule. For example, in some embodiments, error-prone PCR is used. “Error-prone PCR” refers to a process for performing PCR under conditions where the copying fidelity of the DNA polymerase is lowered, such that a high rate of point mutations is obtained along the entire length of the PCR product. See e.g., U.S. Pat. Nos. 5,605,793; 5,811,238; and 5,830,721.

In some embodiments “assembly PCR” is used. “Assembly PCR” refers to a process that involves the assembly of a PCR product from a mixture of small DNA fragments. A large number of different PCR reactions occur in parallel in the same vial, with the products of one reaction priming the product off another. See e.g., U.S. Pat. No. 6,806,048.

In some embodiments, “DNA shuffling” is used. “DNA shuffling” refers to forced homologous recombination between DNA molecules of different but highly related DNA sequences in vitro, caused by random fragmentation of the DNA molecule based on sequence homology, followed by fixation of the crossover by primer extension. See e.g., WO 00/42561 A3 and WO 01/70947 A3.

In some embodiments, sequences derived from introns are used to mediate specific cleavage and ligation of discontinuous nucleic acid molecules to create libraries of novel genes and gene products as described in U.S. Pat. Nos. 5,498,531, and 5,780,272.

In some embodiments, libraries containing ribonucleic acids encoding a novel gene product or novel gene products are created by mixing splicing constructs containing an exon and 3′ and 5′ intron fragments. See e.g., U.S. Pat. No. 5,498,531.

In other embodiments, DNA sequence libraries are created by mixing DNA/RNA hybrid molecules that contain intron-derived sequences that are used to mediate specific cleavage and ligation of the DNA/RNA hybrid molecules such that the DNA sequences are covalently linked to form novel DNA sequences as described in U.S. Pat. No. 6,150,141; WO 00/40715 and WO 00/17342.

In some embodiments, multiple amplification reactions with pooled oligonucleotides, containing mutant protein sequences created by the assembly of gene fragments generated from a nucleic acid template are used. See e.g., U.S. Pat. No. 6,403,312.

Examples of other suitable mutagenesis techniques, include, but are not limited to, exon shuffling (see U.S. Pat. No. 6,365,377; Kolkman & Stemmer (2001) Nat. Biotechnol. 19:423-428), family shuffling (see Crameri, et al. (1998) Nature 391:288-291; U.S. Pat. No. 6,376,246), RACHITT™ (Coco, et al. (2001) Nat. Biotechnol. 19:354-359; WO 02/06469 A2), STEP and random priming of in vitro recombination (see Zhao, et al. (1998) Nat. Biotechnol. 16:258-261; Shao, et al. (1998) Nucl. Acids Res. 26:681-683); exonucleases-mediated gene assembly (U.S. Pat. Nos. 6,352,842 and 6,361,974), GENE SITE SATURATION MUTAGENESIS™ (U.S. Pat. No. 6,358,709), GENE REASSEMBLY™ (U.S. Pat. No. 6,358,709) and SCRATCHY (Lutz, et al. (2001) Proc. Natl. Acad. Sci. USA 98:11248-11253), DNA fragmentation methods (Kikuchi, et al. (1999) supra), and single-stranded DNA shuffling (Kikuchi, et al. (2000) supra).

Although these methods are intended to introduce random mutations throughout the gene, those skilled in the art will appreciate that specific regions of the gene can be mutated, and others left untouched, either by isolating and combining the mutated region with the unmodified region (for example, by cassette mutagenesis; see WO 01/75767 A2; Kim & Mass (2000) Biotechniques 28:196-198; Lanio & Jeltsch (1998) Biotechniques 25:958-965; Ge & Rudolph (1997) Biotechniques 22:28-30; Ho, et al. (1989) Gene 77:51-59), or via in vitro or in vivo recombination (see e.g., WO 02/10183 A1; Abécassis, et al. (2000) Nucl. Acids Res. 28:e88).

In addition to the PCR methods outlined herein, other amplification and gene synthesis methods can be used to generate the libraries of mutant proteins. For example, the library genes can be “stitched” together using pools of oligonucleotides with polymerases (and optionally or solely) ligases. These resulting variable sequences can then be amplified using any number of amplification techniques, including, but not limited to, polymerase chain reaction (PCR), strand displacement amplification (SDA), nucleic acid sequence-based amplification (NASBA), ligation chain reaction (LCR) and transcription-mediated amplification (TMA). In addition, there are a number of variations of PCR which can also find use in the invention, including quantitative competitive PCR (QC-PCR), arbitrarily-primed PCR (AP-PCR), immuno-PCR, Alu-PCR, PCR single-strand conformational polymorphism (PCR-SSCP), reverse transcriptase PCR (RT-PCR), biotin-capture PCR, vectorette PCR, panhandle PCR, and PCR-select cDNA subtraction, among others. Furthermore, by incorporating the T7 polymerase initiator into one or more oligonucleotides, IVT amplification can be performed.

In addition to the other amplification and gene synthesis methods outlined above, libraries of mutant proteins can be generated using chemical mutagenesis, random insertion and deletion, and UV mutagenesis.

The library proteins can be produced by culturing a host cell transformed with a nucleic acid molecule, preferably an expression vector containing a nucleic acid encoding a library protein, under the appropriate conditions to induce or cause expression of the library protein. The conditions appropriate for library protein expression will vary with the choice of the expression vector and the host cell, and can be ascertained by one skilled in the art through routine experimentation. For example, the use of constitutive promoters in the expression vector requires optimizing the growth and proliferation of the host cell, while the use of an inducible promoter requires the appropriate growth conditions for induction. In addition, in some embodiments, the timing of the harvest is important. For example, the baculovirus systems used in insect cell expression are lytic viruses, and thus harvest time selection can be crucial for product yield.

A wide variety of appropriate host cells can be used to produce and screen the mutant libraries, including yeast, bacteria, archaebacteria, fungi, insect, plant and animal cells, including mammalian cells. Of particular interest are Drosophila melanogaster cells, Saccharomyces cerevisiae and other yeasts, E. coli, Bacillus subtilis, Streptococcus cremoris, Streptococcus lividans, SF9 cells, C129 cells, 293 cells, Neurospora, BHK, CHO, COS, and HeLa cells, fibroblasts, Schwanoma cell lines, immortalized mammalian myeloid and lymphoid cell lines, Jurkat cells, mast cells and other endocrine and exocrine cells, and neuronal cells. See e.g., the ATCC cell line catalog. In some embodiments, the cells can be genetically engineered to contain exogenous nucleic acid, for example, to contain target molecules.

Several commercial sources are available for this including, but not limited, to Roche RAPID TRANSLATION SYSTEM™, PROMEGA® TNT® system, the NOVAGEN® ECOPRO™ system, the AMBION® PROTEINSCRIPT-PRO™ system. In vitro translation systems derived from both prokaryotic (e.g., E. coli) and eukaryotic (e.g., Wheat germ, Rabbit reticulocytes) cells are available and can be selected based on the expression levels and functional properties of the protein of interest. Both linear (as derived from a PCR amplification) and circular (as in plasmid) DNA molecules are suitable for such expression as long as they contain the gene encoding the protein operably linked to an appropriate promoter. Other features of the DNA molecule that are important for optimal expression in either the bacterial or eukaryotic cells (including the ribosome binding site etc) are also included in these constructs. The proteins can again be expressed individually or in suitable size pools containing multiple library members. The main advantage offered by the in vitro systems is their speed and ability to produce soluble proteins. In addition, the protein being synthesized can be selectively labeled if needed for subsequent functional analysis.

Methods of introducing exogenous nucleic acid molecules into host cells is well-known in the art, and will vary with the host cell used. Techniques include dextran-mediated transfection, calcium phosphate precipitation, calcium chloride treatment, POLYBRENE®-mediated transfection, protoplast fusion, electroporation, viral or phage infection, encapsulation of the polynucleotide(s) in liposomes, and direct microinjection of the DNA into nuclei. In the case of mammalian cells, transfection can be either transient or stable.

A variety of recombinant expression vectors can be utilized to express the library of proteins. Examples of suitable vectors include, but are not limited to, pED (commercially available from NOVAGEN®), pBAD and pCNDA (commercially available from INVITROGEN™), pEGEX (commercially available from Amersham Biosciences), pQE (commercially available from QIAGEN®). The choice of the appropriate vector can be ascertained by one of skill in the art. Expression vectors embrace self-replicating extrachromosomal vectors or vectors which integrate into a host genome. Expression vectors used in the methods described herein typically contain a library member, control or regulatory sequences, selectable markers, and/or additional elements, such as a purification tag.

The libraries of the invention can be screened, e.g., using a yeast two-hybrid system as exemplified herein and by Chen, et al. ((2004) J. Biol. Chem. 279:33855-33864); Schwimmer, et al. ((2004) Proc. Natl. Acad. Sci. USA 101:14707-14712); and Doyle, et al. ((2001) Chem. Soc. 123:11367-11371). Yeast-based two-hybrid systems utilize chimeric genes and detect protein-protein interactions via the activation of reporter-gene expression. Reporter-gene expression occurs as a result of reconstitution of a functional transcription factor caused by the association of fusion proteins encoded by the chimeric genes. See also, Ausubel, et al., Current Protocols in Molecular Biology, John Wiley & Sons, pp.13.14.1-13.14.14; Sambrook & Russell, Molecular Cloning, Cold Spring Harbor Laboratory Press, 3^{rd} edition, Chapter 18. In addition to the yeast two-hybrid systems, yeast one-hybrid systems, yeast three-hybrid systems, bacterial two-hybrid systems, or mammalian two-hybrid systems can be used.

In some embodiments, host cells other than yeast are used to identify or select novel mutant proteins of interest. Suitable host cells are described herein. As a specific example, HEC-1 cells are transformed with a library representing mutants of a protein and the fold activation in the presence of the target molecule as compared to the wild-type protein is measured.

In some embodiments, other selection or screening methods are used to identify mutant proteins with novel or altered functions. For example, cell-based screening methods based on cell survival, cell death, or expression of reporter genes in cells are used. The screens can employ cells containing individual variants or pools of variants belonging to a library.

In some embodiments, libraries of mutant proteins are attached to or bound to an insoluble support having isolated sample receiving areas (e.g., a microtiter plate, an array, etc.) so that in vitro-based screening approaches can be employed (e.g., binding or activity assays). The insoluble support can be made of any composition to which the assay component can be bound, is readily separated from soluble material, and is otherwise compatible with the overall method of screening. The surface of such supports can be solid or porous and of any convenient shape. Examples of suitable insoluble supports include microtiter plates, arrays, membranes and beads. These are typically made of glass, plastic (e.g., polystyrene), polysaccharides, nylon or nitrocellulose, TEFLON®, etc. Microtiter plates and arrays are especially convenient because a large number of assays can be carried out simultaneously, using small amounts of reagents and samples.

Alternatively, bead-based assays are used, particularly with use in fluorescence-activated cell sorting (FACS). The particular manner of binding the assay component is not crucial so long as it is compatible with the reagents and overall methods described herein, and maintains the activity of the composition.

The library of proteins can be purified or isolated after expression. Library proteins can be isolated or purified in a variety of ways known to those skilled in the art depending on what other components are present in the sample. The degree of purification necessary can vary depending on the use of the library protein. In some instances no purification will be necessary. For example, in some embodiments, if library proteins are secreted, screening or selection can take place directly from the media.

Standard purification methods include electrophoretic, molecular, immunological and chromatographic techniques, including ion exchange, hydrophobic, affinity, size-exclusion chromatography, and reversed-phase HPLC chromatography, as well as precipitation, dialysis, and chromatofocusing techniques. Purification can often be facilitated by the inclusion of purification tag. The choice of the appropriate purification tag can be ascertained by one skilled in the art. For example, the library protein can be purified using glutathione resin if a GST fusion is employed, Immobilized Metal Affinity Chromatography (IMAC) if a His or other tag is employed, or immobilized anti-FLAG® antibody if a FLAG® tag is used. Ultrafiltration and diafiltration techniques, in conjunction with protein concentration, are also useful. For general guidance in suitable purification techniques, see Scopes (1994) Protein Purification: Principles and Practice, 3rd Ed., Springer-Verlag, NY.

The instant method constitutes a conceptually simple and readily generalizable method for significantly altering the selectivity of proteins for a target molecule. This approach involves screening very manageably sized mutant protein libraries and is sensitive to the detection of variants enhanced in target molecule selectivity.

The invention is described in greater detail by the following non-limiting examples.

### EXAMPLE 1

### Nuclear Hormone Receptors

The method described herein is useful for generating and selecting for proteins with novel or altered functions, e.g., orthogonal receptor-ligand pairs. In some embodiments, nuclear hormone receptors are used for the generation of orthogonal receptor-ligand pairs. By way of illustration, suitable nuclear receptors for use in the methods and compositions of the present invention include, but are not limited to, human estrogen receptor alpha (hERα; SEQ ID NO:2) or beta (hERβ; SEQ ID NO:6) proteins or an estrogen receptor alpha protein from Acanthopagrus schlegelii (SEQ ID NO:7), Alligator mississippiensis (SEQ ID NO:8), Astatotilapia burtoni (SEQ ID NO:9), Bos taurus (SEQ ID NO:10), Caiman crocodilus (SEQ ID NO:11), Cavia porcellus (SEQ ID NO:12), Chrysophrys major (SEQ ID NO:13), Coturnix japonica (SEQ ID NO:14), Danio rerio (SEQ ID NO:15), Equus caballus (SEQ ID NO:16), Fundulus heteroclitus (SEQ ID NO:17), Halichoeres tenuispinis (SEQ ID NO:18), Halichoeres trimaculatus (SEQ ID NO:19), Ictalurus punctatus (SEQ ID NO:20), Micropterus salmoides (SEQ ID NO:21), Mus musculus (SEQ ID NO:22), Ovis aries (SEQ ID NO:23), Oncorhynchus masou (SEQ ID NO:24), Paralichthys olivaceus (SEQ ID NO:25), Sparus aurata (SEQ ID NO:26), Taeniopygia guttata (SEQ ID NO:27), Tilapia nilotica (SEQ ID NO:28), and Xenopus laevis (SEQ ID NO:29). In general, members of this superfamily have three modular structural domains, an amino-terminal ligand-independent transactivation domain, a central DNA binding domain (DBD), and a carboxy-terminal ligand binding domain (LBD). See Tables 1 and 2 for domains of hERα and hERβ, respectively).

TABLE 1

| | Position | Position Within |
|---|---|---|
| | Within HERα | HERα Coding |
| Domain | Protein^{a} | Region^{b} |
| Activation Domain 1 (AF-1) | 1-179 | 1-537 |
| DNA Binding Domain (DBD) | 180-262 | 538-786 |
| Hinge Domain | 263-301 | 787-903 |
| Ligand Binding Domain | 302-552 | 904-1656 |
| Activation Domain 2 (AF-2) | Spread out | Spread out |
| | within LBD³ | within LBD³ |
| F-Domain | 553-595 | 1657-1785 |

^{a}Position is in reference to SEQ ID NO: 2.
^{b}Position is in reference to SEQ ID NO: 1.
^{c}Nilsson et al. (2001) supra.

TABLE 2

| | Position | Position Within |
|---|---|---|
| | Within HERβ | HERβ Coding |
| Domain | Protein^{a} | Region |
| Activation Domain 1 (AF-1) | 1-143 | 1-429 |
| DNA Binding Domain (DBD) | 144-226 | 430-678 |
| Hinge Domain | 227-254 | 679-762 |
| Ligand Binding Domain | 255-504 | 763-1512 |
| Activation Domain 2 (AF-2) | Spread out | Spread out |
| | within LBD^{b} | within LBD^{b} |
| F-Domain | 505-530 | 1513-1590 |

^{a}Position is in reference to SEQ ID NO: 6.
^{b}Position is in reference to SEQ ID NO: 5.
^{c}Nilsson et al. (2001) supra.

### EXAMPLE 2

### Estrogen Receptor Alpha Mutants which Bind DHB

Libraries were created by 1) identify all ligand-contacting residues in the receptor structure, 2) performing individual site saturation mutagenesis of all or a subset of these selected residues, 3) screening each library in 96-well plates, 4) selecting the mutant most selective for the target ligand relative to the natural ligand, 5) performing a second round of individual site saturation mutagenesis at the remaining unmutated ligand-contacting residues, 6) repeating steps 3-5 until no further improvement can be achieved, and 7) performing random mutagenesis on the whole receptor followed by library screening to isolate mutants with mutations that are not within the ligand binding pocket and yet affect ligand selectivity.

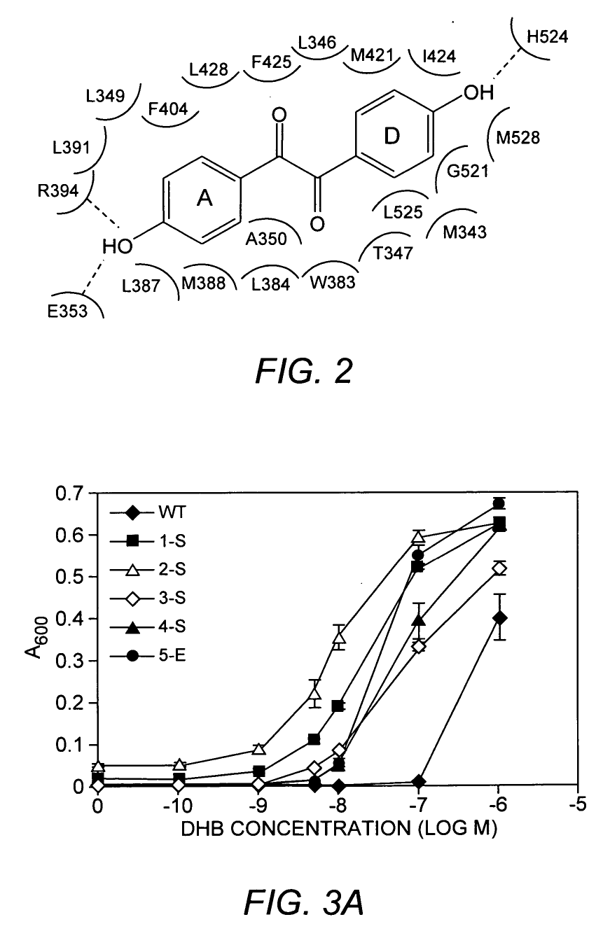

Twenty-one residues were identified to be in direct contact (within 4.6 Å) with the docked DHB ligand (FIG. 2). To reduce the load for screening, Arg394, Glu353, and His524 were left unchanged, because of their known role in hydrogen bonding with the terminal hydroxyl groups of the ligand; residues Leu349, Leu387, Phe404, and Leu392, which contact the A-ring portion of the ligand forming a tightly maintained ligand-binding subpocket restricting the conformational flexibility of the A-ring were similarly left unchanged (Anstead, et al. (1997) Steroids 62:268-303). Thus, 14 residues in total were selected for individual site saturation mutagenesis. For each site, only 32 distinct library variant possibilities existed (32 possible codon substitutions). The screening of 95 library transformants per randomized site in a convenient 96-well plate format (or 190 transformants per site, as done here) provided comprehensive coverage of the created variants.

Phenotypic screening of library variants was carried out based on a yeast two-hybrid system employing two constructs, the hERα LBD construct fused to the DNA binding domain of the yeast Gal4 transactivator, and the common mammalian transcriptional coactivator steroid receptor coactivator-1 (SRC-1) fused to the yeast Gal4 transcriptional activation domain. The hERα-SRC-1 interaction, which is elemental in the role of hERα as a transcriptional activator, is strengthened by the binding of agonist ligands to hERα. This system couples the strength of ligand-receptor interaction within host yeast cells to their growth on media lacking histidine and can be applied in either a selection or screening mode (Chen, et al. (2004) supra).

Variants with increased response to DHB relative to the parental construct were selected based on growth of the host yeast cells on agar plates lacking histidine and containing an appropriate concentration of DHB. The selected mutants were subsequently assayed against both DHB and the natural hERα ligand, 17β-estradiol (E₂), in a cell growth-based 96-well plate assay to ensure sufficient selectivity. Transformants were individually picked from non-selective (with histidine, without DHB) growth media plates, and assayed for cell growth-based response to both target ligand (look for strengthened response) and natural ligand (look for weakened response) in 96-well plates. This phenotypic screening approach can also be applied to libraries created by individual site-saturation mutagenesis. The selection-based approach, using growth in yeast cells, is useful for screening large libraries of variants created using error-prone PCR-based random point mutagenesis.

Mutants leading to increased or unchanged growth in DHB-containing media and exhibiting decreased growth in E₂-containing media relative to the parental mutant were visually identified and subjected to a growth-based ligand dose-response assay in yeast cells. The plasmids from promising mutants based on this ligand response assay were isolated and re-transformed into fresh yeast cells, and the ligand response assay was carried out again to eliminate possible false-positives.

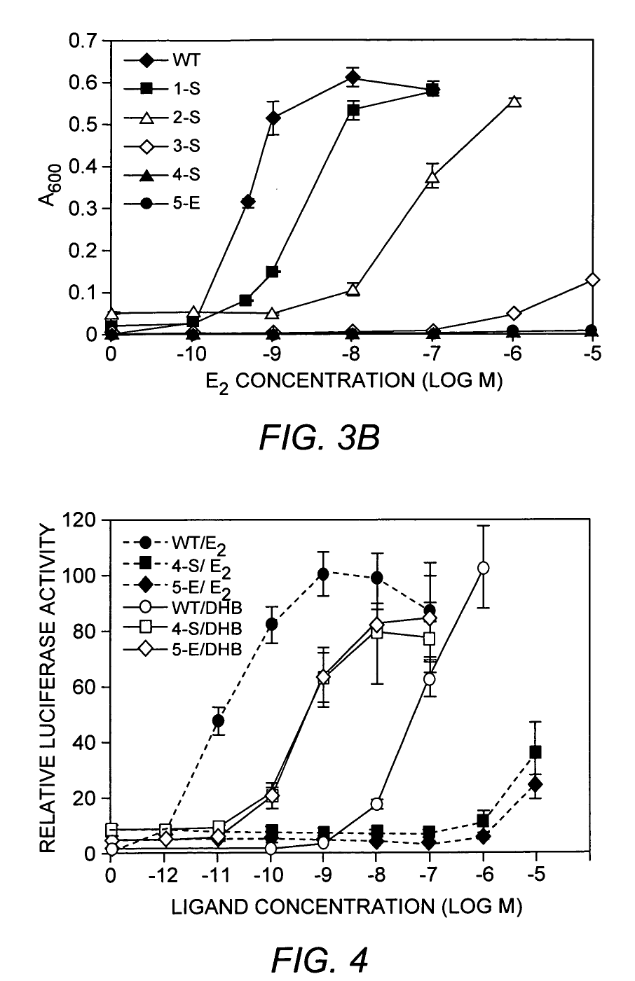

In total, four rounds of individual site-saturation mutagenesis and one round of error-prone polymerase chain reaction (PCR)-based random point mutagenesis were performed. One hundred and ninety transformants were picked from each saturation mutagenesis library and assayed in 96-well plates. For the random mutagenesis library, 3.3×10⁶ transformants were subjected to selection, and 1900 colonies appearing on selective agar growth plates were picked and assayed in 96-well plates. In each round, a number (ranging from 1-6) of DHB-selective mutants were identified, the most selective of which was picked and carried forth to the next round of mutagenesis and screening. It should be noted that in cases where more than one DHB-selective mutant was found in a given round of mutagenesis, these mutants appeared in libraries for different randomized sites. The yeast two-hybrid dose responses and corresponding ligand concentrations leading to half-maximal response (EC₅₀) of the best mutants identified at each round of screening are presented in FIG. 3A, FIG. 3B and Table 3.

TABLE 3

| | | EC_{50, DNB} | EC_{50, C2} | | Fold |
|---|---|---|---|---|---|
| Round | Mutation | (nM) | (nM) | Selectivity | Improvement |
| Wild-Type | - | 500 ± 200 | 0.5 ± 0.3 | 1.0 × 10⁻³ | 1.0 |
| 1-S | Ala350Met | 25 ± 20 | 3.0 ± 2.2 | 0.1 | 1.0 × 10² |
| 2-S | Ala350Met | 10 ± 5 | 70 ± 30 | 7.0 | 7.0 × 10³ |
| | Leu346Ile | | | | |
| 3-S | Ala350Met | 100 ± 80 | ≧5000 | ≧50 | ≧5.0 × 10⁴ |
| | Leu346Ile | | | | |
| | Met388Gln | | | | |
| 4-S | Ala350Met | 65 ± 40 | ≧65000^{‡} | ≧1.0 × 10^{†} | ≧1.0 × 10^{6†} |
| | Leu346Ile | | | | |
| | Met388Gln | | | | |
| | Gly521Ser | | | | |
| | Tyr526Asp | | | | |
| 5-E | Ala350Met | 100 ± 40 | ≧10^{6‡} | ≧1.0 ×°10^{\4} | ≧1.0 × ^{7†} |
| | Leu34GIle | | | | |
| | Met388Gln | | | | |
| | Gly521Ser | | | | |
| | Tyr526Asp | | | | |
| | Phe461Leu | | | | |
| | Val560Met | | | | |

†based on incubation of yeast two-hybrid ligand response microtiter plates at room temperature for 3-4 days, after which time mutants responded to high concentrations (≧1μM) of E₂.
‡Values calculated from the estimated selectivity (^{†}) and EC₅₀ values for 4,4′-dihydroxybenzil (DHB)

Mammalian cell transactivation profiles for the wild-type hERα and the two best mutants, 4-S and 5-E, were carried out in estrogen receptor-negative human endometrial cancer (HEC-1) cells after cloning the hERα LBD from the chimeric yeast two-hybrid construct into the full-length estrogen receptor construct. Dose responses from this analysis are presented in FIG. 4 and the corresponding EC₅₀ values are presented in Table 4.

TABLE 4

| | | EC_{50, DHB} | EC_{50, E2} | | Fold |
|---|---|---|---|---|---|
| Round | Mutation | (nM) | (nM) | Selectivity | Improvement |
| Wild-Type | - | 66 ± 19 | 0.012 | 1.8 × 10⁻⁴ | 1.0 |
| 1-S | Ala350Met | n.d. | n.d. | n.d. | n.d. |
| 2-S | Ala350Met | n.d. | n.d. | n.d. | n.d. |
| | Leu346Ile | | | | |
| 3-S | Ala350Met | n.d. | n.d. | n.d. | n.d. |
| | Leu346Ile | | | | |
| | Met388Gln | | | | |
| 4-S | Ala350Met | 0.37 ± 0.02 | ≧1.0 × 10⁴ | ≧2.7 × 10⁴ | ≧1.5 × 10⁸ |
| | Leu346Ile | | | | |
| | Met388Gln | | | | |
| | Gly521Ser | | | | |
| | Tyr526Asp | | | | |
| 5-E | Ala350Met | 0.38 ± 0.17 | ≧1.0 × 10⁴ | ≧2.6 × 10⁴ | ≧1.4 × 10⁸ |
| | Leu346Ile | | | | |
| | Met388Gln | | | | |
| | Gly521Ser | | | | |
| | Tyr526Asp | | | | |
| | Phe461Leu | | | | |
| | Val560Met | | | | |

†Estimates based on incubation of yeast two-hybrid ligand response microtiter plates at room temperature for 3-4 days, after which time mutants responded to high concentrations (≧1μpM) of E₂.
‡Values calculated from the estimated selectivity (^{†}) and EC₅₀ values for 4,4′-dihydroxybenzil (DHB).

Thus, by combining stepwise, targeted site-saturation mutagenesis of ligand-contacting protein residues and random point mutagenesis with phenotypic screening or selection in a yeast two-hybrid system, hERα specificity for the synthetic ligand (DHB) versus the natural ligand (E₂) was shifted by more than 10⁷-fold. The resulting ligand-receptor pair was highly sensitive to DHB in mammalian cells and was almost fully orthogonal to the natural ligand-receptor pair. Notably, 3 of the 4 substitutions created in the ligand binding pocket (Ala350Met, Leu346Ile, Met388Gln), contributing a combined target ligand selectivity improvement of ≧5×10⁴-fold relative to the wild-type hERα (Tables 3 and 4), could not have been obtained through single base pair substitutions.

In contrast to the expectation that a predominantly polar binding pocket would be required to complement the polar α-dicarbonyl core of DHB, much of the engineered selectivity was derived from variations in hydrophobicity. This observation underlines the potential drawbacks of limiting the amino acids available for substitution at particular receptor sites based on rational considerations.

To understand the potential role played by the Ala350Met mutation by modeling, the substitution (following energy minimization of all binding pocket and surrounding residues) was made to the docked DHB-hERα complex. This analysis revealed that the extended hydrophobic side chain of methionine makes a favorable hydrophobic contact with the D-ring analogue of DHB, whereas the short side chain of alanine cannot make this contact. In addition to this favorable hydrophobic interaction, the sulfur atom of the methionine is within 6 Å of carbon atoms in both the A-ring and D-ring of DHB, resulting in potentially favorable sulfur-aromatic dispersion interactions (Reid, et al. (1985) FEBS Letters 190:209-213). Moreover, the long side chain of methionine might clash with the bulky hydrophobic core of E₂, leading to a weakened E₂ response. A similar analysis to gauge the effect of the Met388Gln mutation indicates that glutamine could donate a hydrogen bond to one of the ketone moieties of DHB. The accompanying unfavorable interaction with E₂ was presumably due to the introduction of a polar side-group into direct contact with the hydrophobic core of E₂. Thus, both of these substitutions appeared to make dual contributions to the shift in ligand binding selectivity, enhancing the stability of DHB binding while disabling E₂ binding.

It should be noted that in rounds 4 and 5 of mutagenesis and screening, two mutations were introduced into the best-identified mutants (mutants 4-S and 5-E). In the fourth round, the non-binding pocket mutation (Tyr526Asp) was the result of a point mutation introduced during polymerase amplification. Site-directed mutagenesis to separate the contributions of Gly521Ser and Tyr526Asp in mutant 4-S revealed that Gly521Ser was primarily responsible for the observed selectivity enhancement relative to mutant 3-S. It was found that in the absence of the Tyr526Asp mutation, a significant amount of basal level ligand-independent response was present. This indicated that the Tyr526Asp mutation (positioned on helix 11) directly or indirectly influenced the conformation of helix 12, which contains a ligand-dependent activation function (AF-2) in hERα. In mutant 5-E, site-directed mutagenesis experiments revealed that the observed selectivity enhancement in yeast cells (Table 3) relative to mutant 4-S was entirely due to the Phe461Leu mutation, and that Val560Met had no detectable effect. Residue 461 was distant from the ligand binding pocket.

For the most part, the ligand selectivity displayed by the chimeric hERα mutants in yeast cells was reproduced well by the full-length constructs in mammalian cells (Table 4). The EC₅₀ values in mammalian cells were, in fact, lower than the corresponding values in yeast cells; this phenomenon has been observed previously (Schwimmer, et al. (2004) supra; Chen, et al. (2004) supra), and is probably related to the increased permeability of the ligands for entry into mammalian cells. Overall, the ligand selectivities of the mutants in yeast and mammalian cells correlate with each other well, with the mutants being actually more selective for DHB compared to E₂ in HEC-1 cells than in yeast two-hybrid cells (Tables 3 and 4).

The fifth round mutant (5-E) appeared to show no selectivity enhancement relative to the fourth round mutant (4-S) in either yeast (see FIG. 3) or in mammalian cells (see FIG. 4). In yeast, the estimated selectivity difference (Table 3) arose primarily from a weakened E₂ response compared to the 4-S construct, observed after extended incubation of the ligand response assay plates. In mammalian cells, this weakened E₂ response was not apparent. This disparity between the yeast and mammalian cell systems might be related to the presence of numerous interacting co-activators in mammalian cells compared to the single SRC-1 co-activator that was introduced for the assays in yeast. These additional co-activators, unlike SRC-1, might not be able to distinguish between the E₂-bound mutants 4-S and 5-E.

The best receptor variant obtained after four rounds of individual site-saturation mutagenesis and one round of error-prone PCR, i.e., 5-E, despite being highly selective for DHB compared to E₂, did not respond to DHB with a potency fully equivalent to that of the wild-type hERα-E₂ response. To enhance the ligand response potency for DHB, further rounds of error-prone PCR mutagenesis and selection based on mutant 5-E were performed. Despite subjecting a library of 2.4×10⁶ transformants to yeast two-hybrid selection, no variants with significantly improved potency or selectivity for DHB were found. Not wishing to be bound by theory, it was believed that the inability to identify mutants more sensitive for DHB was be due to the inability of error-prone PCR to access important amino acid substitutions from single base pair changes.

Accordingly, engineering efforts to strengthen the DHB response of mutant 4-S were focused on saturation mutagenesis of individual sites. Mutagenesis was carried out by taking into consideration the following six sites located outside the ligand binding pocket which were known to be important for ligand sensitivity, namely amino acid residues located at position 442, 536, 537, 459, 466 and 534 of SEQ ID NO:2 (Chen et al. (2004) supra); and the following additional sites within the binding pocket, namely amino acid residues located at position 349, 387, 391, 404 and 524 of SEQ ID NO:2. Thus, 11 sites in the hERα-LBD were subjected to site-directed mutagenesis and EP-PCR as described herein.

In the first round of mutagenesis and screening, based on the mutant 4-S template, one mutant (5-S) was found with a ˜10-fold strengthened response to DHB and similarly strengthened response to E₂. The yeast two-hybrid dose response analysis for this mutant and the parental mutant 4-S toward both DHB and E₂ are listed in Table 5.

TABLE 5

| | | OD₆₀₀ ± Std. Error | |
|---|---|---|---|
| Ligand | Concentration | 4-S Parent | 5-S Mutant |
| DHB | 1.00E−11 | 0.0011 ± 0.0001 | 0.0006 ± 0.0002 |
| | 1.00E−10 | 0.0009 ± 0.0002 | 0.0006 ± 0.0006 |
| | 1.00E−09 | 0.0014 ± 0.0001 | 0.0194 ± 0.0058 |
| | 5.00E−09 | 0.0005 ± 0.0004 | 0.2806 ± 0.0414 |
| | 1.00E−08 | 0.055 ± 0.0035 | 0.4114 ± 0.0418 |
| | 1.00E−07 | 0.4001 ± 0.0171 | 0.6848 ± 0.0294 |
| | 1.00E−06 | 0.6995 ± 0.0205 | 0.7322 ± 0.0088 |
| E₂ | 1.00E−10 | 0.0006 ± 0.0002 | 0.0006 ± 0.0002 |
| | 1.00E−09 | 0.0005 ± 0.0005 | 0.0003 ± 5E−05 |
| | 1.00E−08 | 0.0023 ± 0.0011 | 0.0009 ± 0 |
| | 1.00E−07 | 0 ± 0 | 0.0005 ± 0.0005 |
| | 1.00E−06 | 0.0006 ± 0.0006 | 0.0003 ± 0 |
| | 1.00E−05 | 0.0018 ± 0.0014 | 0.3778 ± 0.0534 |

Sequencing of mutant 5-S revealed one additional mutation relative to the mutant 4-S template, namely Gly442Tyr. In the subsequent round of mutagenesis and screening, mutant 5-S was held fixed, and the remaining unmutated sites within and outside of the ligand binding pocket (20 sites total: 5 from outside the binding pocket, and 15 from within the binding pocket, including the 10 unmutated sites from Example 2 and positions 349, 387, 391, 404 and 524) were subjected to individual site saturation mutagenesis. From this library, one mutant (6-S) with a ˜2-fold strengthened response to both DHB and E₂ was identified. The dose response analysis for this mutant and the parental mutant 5-S in yeast cells are presented in Table 6.

TABLE 6

| | | OD₆₀₀ ± Std. Error | |
|---|---|---|---|
| Ligand | Concentration | 5-S Parent | 6-S Mutant |
| DHB | 1.00E−11 | 0.0006 ± 0.0002 | 0.0013 ± 0.0001 |
| | 1.00E−10 | 0.0006 ± 0.0006 | 0.0016 ± 0.0002 |
| | 1.00E−09 | 0.0194 ± 0.0058 | 0.0863 ± 0.0183 |
| | 5.00E−09 | 0.2806 ± 0.0414 | 0.3479 ± 0.0249 |
| | 1.00E−08 | 0.4114 ± 0.0418 | 0.5021 ± 0.043 |
| | 1.00E−07 | 0.6848 ± 0.0294 | 0.6907 ± 0.0347 |
| | 1.00E−06 | 0.7322 ± 0.0088 | 0.7277 ± 0.0267 |
| E₂ | 1.00E−10 | 0.0006 ± 0.0002 | 0.0013 ± 1E−04 |
| | 1.00E−09 | 0.0003 ± 5E−05 | 0.0021 ± 0 |
| | 1.00E−08 | 0.0009 ± 0 | 0.0014 ± 0.0005 |
| | 1.00E−07 | 0.0005 ± 0.0005 | 0 ± 0 |
| | 1.00E−06 | 0.0003 ± 0 | 0.0036 ± 0.0003 |
| | 1.00E−05 | 0.3778 ± 0.0534 | 0.4430 ± 0.0256 |

Sequence analysis of mutant 6-S revealed one additional mutation relative to the mutant 5-S template, namely Leu466Ser.

By combining straightforward selection of target protein residues with the power of directed evolution, the selectivity of a natural nuclear hormone receptor, hERα, for a synthetic ligand DHB was improved by more than 10⁷-fold compared to the natural ligand E₂, relative to the wild-type hER. The resulting hERα mutant responded to subnanomolar concentrations of DHB in mammalian cells and was essentially unresponsive to E₂, thus being essentially orthogonal to the wild-type hERα-E₂ combination. Accordingly, particular embodiments embrace a mutant human estrogen receptor alpha protein which efficiently binds DHB as compared to wild-type protein. Mutants embraced by this embodiment include hER variants containing one or more of the following mutations relative to SEQ ID NO:2; Ala350Met, Leu346Ile, Met388Gln, Gly521Ser, Tyr526Asp, Phe461Leu, Val560Met, Gly442Tyr, and Leu466Ser.

### EXAMPLE 3

### Estrogen Receptor Alpha Mutants which Bind L9

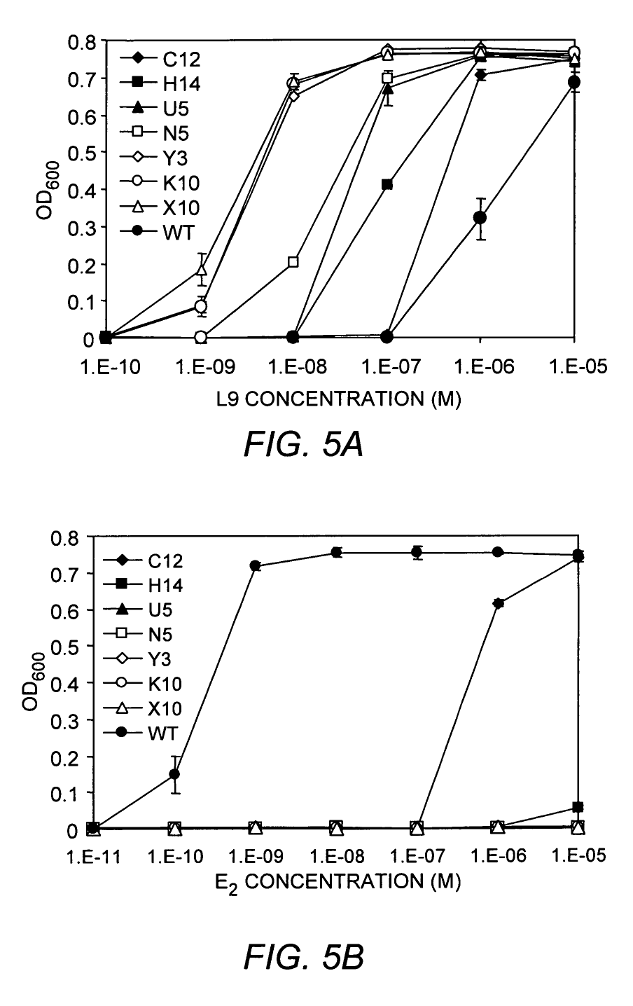

Using the same approach to generate mutant estrogen receptors which bind DHB, six rounds of stepwise site saturation mutagenesis were performed on the hERα-LBD toward the target synthetic ligand 2,4-di(4-hydroxyphenyl)-5-ethylthiazole (L9) (Fink, et al. (1999) Chem. Biol. 6:205-19). The transactivation profiles and EC₅₀ values of exemplary mutants found in each round of screening, as well as that of the wild-type hERα, are presented in FIG. 5A, FIG. 5B and Table 7.

TABLE 7

| | | | EC₅₀ | EC₅₀ | | |
|---|---|---|---|---|---|---|
| | Mutant | | (L9), | (E₂), | | Fold |
| Round | Name | Mutation | (nM) | (nM) | Selectivity | Improvement |
| 0 | Wild- | None | 2300 | 0.3 | 0.000130 | 1 |
| | Type | | | | | |
| 1-S | C12 | Gly521Thr | 450 | 500 | 1.11 | 8518 |
| 2-S | H14 | Gly521Thr | 90 | >10⁴ | >111 | >8.51 × 10⁵ |
| | | His524Tyr | | | | |
| 3-S | U5 | Gly521Thr | 42 | >10⁴ | >238 | >1.82 × 10⁶ |
| | | His524Tyr | | | | |
| | | Met388Phe | | | | |
| 4-S | N5 | Gly521Thr | 20 | >10⁵ | >5000 | >3.83 × 10⁷ |
| | | His524Tyr | | | | |
| | | Met388Phe | | | | |
| | | Thr347Cys | | | | |
| 5-S | Y3 | Gly521Thr | 3.5 | >10⁵ | >28571 | >2.19 × 10⁸ |
| | | His524Tyr | | | | |
| | | Met388Phe | | | | |
| | | Thr347Cys | | | | |
| | | Met528Asp | | | | |
| 6-S | K10 | Gly521Thr | 3.5 | >10⁵ | >28571 | >2.19 × 10⁸ |
| | | His524Tyr | | | | |
| | | Met388Phe | | | | |
| | | Thr347Cys | | | | |
| | | Met528Asp | | | | |
| | | Ile424Val | | | | |
| 7-E | X10 | Gly521Thr | 2.2 | >10⁵ | >45454 | >4.38 × 10⁸ |
| | | His524Tyr | | | | |
| | | Met388Phe | | | | |
| | | Thr347Cys | | | | |
| | | Met528Asp | | | | |
| | | Ile424Val | | | | |
| | | Ala376Val | | | | |
| | | His577Δa† | | | | |

†deletion resulting in frame-shift, wherein the following C-terminal sequence was obtained: LPCKSITSRGRQRVSLPQSEVDSRGSIRPGLEPGSTLEPYSESYYCSQANSGRISYDL (SEQ ID NO:30).
“S” refers to the use of saturation mutagenesis of ligand-contacting residues for protein variant library creation, while “E” refers to error-prone PCR-based mutagenesis.

Upon six rounds of stepwise, individual site saturation mutagenesis on a set of 19 sites within the ligand binding pocket of human estrogen receptor alpha (i.e., 343, 346, 347, 349, 350, 383, 384, 387, 388, 391, 404, 421, 424, 425, 428, 521, 524, 525, and 528 of SEQ ID NO:2), an engineered receptor variant with >10⁸-fold shifted selectivity toward the target ligand L9 was generated. It is contemplated that additional mutagenesis can be applied to the X-10 variant to achieve an EC₅₀ in yeast of <0.03 nM (i.e., 10-fold stronger response than that of the wild-type hERα-LBD toward the natural ligand, E₂).

Thus, additional embodiments of the present invention embrace a mutant human estrogen receptor alpha protein which efficiently binds L9 as compared to wild-type protein. Mutants embraced by this embodiment include hER variants containing one or more of the following mutations relative to SEQ ID NO:2; Gly521Thr, His524Tyr, Met388Phe, Thr347Cys, Met528Asp, Ile424Val, Ala376Val, and His577Δa (wherein the amino acid sequence LPCKSITSRGRQRVSLPQSEVDSRGSIRPGLEPGSTLEPYSESYYCSQANSGRISYDL, SEQ ID NO:30, replaces the C-terminus of hER).

### EXAMPLE 4

### Materials and Methods

Plasmids, Strains, Reagents and Growth Media. The pGAD424-SRC1 ‘prey’ plasmid containing the full-length SRC-1 co-activator was constructed using standard methods (Ding, et al. (1998) Mol. Endocrinol. 12:302-313). A nucleic acid molecule (SEQ ID NO:3) encoding the LBD and F-domain of hERα (SEQ ID NO:4) were inserted downstream of the Gal4 DNA binding domain in the pBD-Gal4-Cam ‘bait’ plasmid (STRATAGENE®, La Jolla, Calif.; Chen, et al. (2004) J. Biol. Chem. 279:33855-33864). The yeast two-hybrid strain YRG2 (STRATAGENE®) was employed. The cloning of hERα LBD mutant constructs into the mammalian expression vector pCMV5 has been described (Chen, et al. (2004) supra). Rich media used for growth of yeast cells was YPAD (Woods & Gietz (2000) Yeast Transformation, Eaton Publishing, Natick, Mass.), while minimal media was SC dropout media lacking the appropriate amino acids (Rose (1987) Meth. Enzymol. 152:481-504). Taq DNA polymerase was obtained from PROMEGA® (Madison, Wis.), and PFUTURBO® DNA polymerase was purchased from STRATAGENE®. 4,4′-Dihydroxybenzil was synthesized using established methods. Unless otherwise specified, all other reagents were obtained from SIGMA-ALDRICH (St. Louis, Mo.).

Library Generation. The procedure used for generating libraries whereby single residues were randomized to all 20 possible amino acids involved overlap extension coupled with polymerase chain reaction (Ho, et al. (1989) Gene 77:51-59). Briefly, four primers were used to generate an amplified gene library composed of a saturation mutagenized residue. Two primers flanked the hERα LBD region CamL-ERa, 5′-CGA CAT CAT CAT CGG AAG AG-3′ (SEQ ID NO:31) and CamR-ERa, 5′-GCT TGG CTG CAG TAA TAC GA-3′ (SEQ ID NO:32) and two exactly complementary degenerate primers incorporating the residue to be mutated (one primer for generating the sense strand, and the other for generating the anti-sense strand). The two degenerate primers incorporating the randomized amino acids substituted the codon corresponding to the target residue with the sequence NNS, and contained 9-10 additional bases on either side (5′ and 3′). The choice of the substitution NNS allowed the incorporation of all 20 amino acids, while keeping the total number of codon possibilities low, at 32. For each gene library containing a randomized codon, four PCR reactions were performed. First, two separate PCR reactions were performed, using the pBD-Gal4-Cam vector harboring the appropriate Gal4-BD-hERα-LBD construct as a template, to amplify a 5′-portion and 3′-portion of the hERα LBD gene containing the NNS-substitution at the codon of interest. Each PCR reaction was a standard reaction containing, in a final volume of 50 μl, 1× Taq DNA polymerase buffer containing 1.5 mM MgCl₂ (PROMEGA®, Madison, Wis.), 0.2 mM dNTPs (Roche, Indianapolis, Ind.), 0.5 μM of appropriate flanking primer (CamL-ERa or CamR-ERa), 0.5 μM of appropriate degenerate primer, 5 ng of template plasmid, 0.6 U Taq DNA polymerase, and 0.6 U PFUTURBO® DNA polymerase. PCR reactions were carried out on a MJ Research (Watertown, Mass.) PTC-200 thermocycler for 25 cycles of 30 seconds at 94° C., 30 seconds at 55° C., and 1 minute at 72° C. Both PCR products from these reactions were isolated from a 1% agarose gel using the QIAEX® II gel purification kit (QIAGEN®, Chatsworth, Calif.) and treated with the restriction enzyme DpnI to remove any residual methylated template from the products. Two nM of each PCR product were then combined in a 20 μl overlap extension reaction without primers. The reaction conditions of this overlap extension were identical to those described for the standard PCR described above, except for the absence of primers and the use of a different program employing 10 cycles of 1 minute at 94° C., 1 minute at 55° C., and 3 minutes at 72° C. Finally, 4 μl of this overlap extension reaction was used as the template for a standard PCR reaction (see description above for conditions) for the amplification of the gene library incorporating a randomized codon, using primers CamL-ERa and CamR-ERa. For generating randomly point-mutated (error-prone PCR) libraries, primers CamL-ERa and CamR-ERa were used to amplify the appropriate parental hERα LBD construct contained in the pBD-Gal4-Cam plasmid. Each PCR reaction contained (100 μl final volume) 1× reaction buffer containing 7 mM MgCl₂, 0.15 mM MnCl₂, 500 mM KCl, 100 mM Tris-HCl (pH 8.3 at 25° C.), 0.1% (weight/volume) gelatin, 0.2 mM dGTP, 0.2 mM dATP, 1 mM dCTP, 1 mM dTTP, 0.5 μM of both primers, 20 ng of template plasmid, and 5 U Taq DNA polymerase (PROMEGA®). PCR reactions were carried out for 15 cycles of 30 seconds at 94° C., 30 seconds at 50° C., and 1 minute at 72° C. PCR products from this reaction were purified from a 1% agarose gel using the QIAEX® II gel purification kit (QIAGEN®, Chatsworth, Calif.).

Library Cloning and Transformation. A 10-bp fragment was removed from the multiple cloning site of pBD-Gal4-Cam by digestion with EcoRI and SalI. For individual site saturation mutagenesis libraries, 20 ng of this gapped expression vector was co-transformed with 20 ng of mutagenized hERα LBD PCR product into YRG2 yeast cells pre-transformed with the pGAD424-SRC1 plasmid using the lithium acetate/single-stranded DNA/polyethylene glycol protocol (Gietz & Woods (2002) Method Enzymol. 350:87-96). In the case of error-prone PCR libraries, 150 ng of gapped expression vector was co-transformed with 150 ng of mutagenized hERα LBD PCR product per single transformation in a 30-fold scaled up large-scale transformation (Gietz & Woods (2002) supra). The two co-transformed linear DNA fragments shared 40-60 bp of homology at their ends, allowing the yeast cells to recombine the linear fragments in vivo, giving rise to a circular plasmid expressing the fusion protein Gal4DBD-hERα-LBD. All saturation mutagenesis library transformations were plated onto SC minimal media agar plates lacking leucine and tryptophan (for selection of the plasmids expressing the pGAD424-SRC1 and pBD-Gal4-Cam plasmids, respectively). Error-prone PCR (and combinatorial site-saturation mutagenesis) library transformations were plated onto SC minimal media agar plates lacking leucine, tryptophan and histidine, and containing appropriately concentrated target ligand (DHB) for screening. In the case of round 5 of mutagenesis and screening, the selection condition chosen for library screening was 2.5×10⁻⁸ M DHB.

Molecular Modeling. Docking of the synthetic ligand DHB into the binding pocket of hERα LBD was performed using Molecular Operating Environment (MOE) (Chemical Computing Group, Montreal, Canada). A model of hERα LBD complexed with the synthetic ligand was built from the hERα-diethylstilbestrol (DES) structure (PDB code 3ERD) : (i) the forcefield MMFF94s (Halgren (1999) J. Comput. Chem. 20:720-729) was applied, (ii) hydrogen atoms were added, (iii) partial charges were assigned to all atoms, and (iv) the structure was subsequently energy minimized using a sequential combination of steepest descent, conjugate gradient, and truncated Newton algorithms (Gill, et al. (1981) Practical Optimization, Academic Press, New York). Subsequently, a docking box with a grid consisting of 47×30×27 points was drawn around the DES ligand to specify the boundaries for the movement of the ligand to be docked. In this orientation, the box included the entire DES ligand and a few atoms of the interacting residues. The DES ligand was subsequently deleted from the structure, and the DHB ligand (which had previously been assigned partial charges and minimized using the MMFF94s force field) was docked into the docking box using a simulated annealing algorithm (Hart & Read (1992) Proteins 13:206-222) with the following parameters: initial temperature 12000 K, 25 runs involving six cycles per run, and 20000 iterations per cycle. The five structures with the best docking score (lowest overall energy) from these docking runs were compared and found to be within a root mean square deviation (RMSD) of 0.5 Å from each other. The lowest energy of these five was then subjected to energy minimization as described earlier, in order to determine the most favorable conformation and orientation of DHB in the ligand binding pocket. Residues within 4.6 Å of the docked DHB were considered to be in contact with the ligand for purposes of receptor engineering. For gauging the individual role played by the Ala350Met and Met388Gln mutations, the appropriate amino acid substitutions were made to the docked DHB-hERα structure, and the resulting structure was energy minimized. For superposition of hERα-bound E₂ and DHB, the energy minimized E₂-hERα crystal structure (PDB code 1GWR) was superimposed upon the docked and energy minimized DHB-hERα structure, using the align function in MOE.

Yeast Two-Hybrid System Based Screening. Transformants from individual site-saturation mutagenesis library plates as well as error-prone PCR library plates were picked with sterile toothpicks and incubated overnight (˜16-20 hours) at 30° C. in round-bottom 96-well plates (Evergreen Scientific, Los Angeles, Calif.) containing 50 μl of SC -Leu/-Trp minimal liquid media in each well. As a control, one well in every microtiter plate was inoculated with a yeast colony expressing the parental hERα LBD construct. After the overnight incubation, 250 μl of sterile ddH₂Q was added to every well, and 5 μl of each diluted culture was then transferred to the corresponding wells of two sterile flat-bottom 96-well microtiter plates (Rainin, Oakland, Calif.) containing 200 μl of SC -Leu/-Trp/-His media with an appropriate concentration of either target ligand (DHB) or 17β-estradiol. Appropriate ligand concentrations for this screening were chosen based on the response of the parental hERα LBD construct. For each round of screening, a DHB concentration was selected at which the parental hERα LBD construct responded weakly or not at all, while the concentration of 17β-estradiol for screening was selected such that the parental construct responded moderately. These ligand-containing microtiter plates were incubated at 30° C. for 24 hours, after which they were visually inspected for identification of mutants with strengthened response toward the target ligand (higher cell density than parental mutant control) and weakened response towards 17β-estradiol (lower cell density than parent). One hundred and ninety mutants were screened per saturation mutagenesis library using this approach, with 95 library variants and one parental construct-expressing yeast being used as a control per microtiter plate.

Ligand Dose Response Assay. Overnight cultures of the appropriate yeast cells were diluted in SC -Leu/-Trp/-His minimal media to a final OD₆₀₀ of 0.002. 190 μl aliquots of this diluted culture were added into the wells of a sterile flat bottom 96-well microtiter plate (Rainin, Oakland, Calif.), followed by the addition of 10 μl of appropriately concentrated ligand composed of a 50-fold dilution of ethanol stock solution in SC -Leu/-Trp/-His minimal media. These microtiter plates were incubated at 30° C. for 24 hours, after which cultures were mixed by pipetting, and OD₆₀₀ readings were taken using a SPECTRAMAX® 340PC plate reader (Molecular Devices, Sunnyvale, Calif.).

Mammalian Transfection and Luciferase Assay. Methods used for cell culture, transfection, and performance of luciferase assay are known in the art (Muthyala, et al. (2003) J. Med. Chem. 46:1589-1602).

What is claimed is:

1. A method for generating a mutant protein which efficiently binds a target molecule comprising
identifying one or more amino acid residues comprising a binding site of a wild-type protein for a target molecule;
subjecting at least one amino acid residue of the binding site to saturation mutagenesis;
selecting for at least one binding site mutant protein with enhanced binding efficiency for the target molecule compared to binding efficiency of the wild-type protein for the target molecule;
subjecting the binding site mutant protein to random mutagenesis; and
selecting for at least one mutant protein with enhanced binding efficiency for the target molecule compared to binding efficiency of the wild-type protein or binding site mutant protein for the target molecule thereby generating a mutant protein which efficiently binds the target molecule.

2. An isolated mutant protein identified by the method of claim 1.


SEQUENCE LISTING (32 sequences)

SEQ ID NO: 1 - DNA, 1788 bp, Homo sapiens
atgaccatgaccctccacaccaaagcatccgggatggccctactgcatcagatccaagggaacgagctggagcccctgaaccgtccgcagctcaagatccccctggagcggcccctgggcgaggtgtacctggacagcagcaagcccgccgtgtacaactaccccgagggcgccgcctacgagttcaacgccgcggccgccgccaacgcgcaggtctacggtcagaccggcctcccctacggccccgggtctgaggctgcggcgttcggctccaacggcctggggggtttccccccactcaacagcgtgtctccgagcccgctgatgctactgcacccgccgccgcagctgtcgcctttcctgcagccccacggccagcaggtgccctactacctggagaacgagcccagcggctacacggtgcgcgaggccggcccgccggcattctacaggccaaattcagataatcgacgccagggtggcagagaaagattggccagtaccaatgacaagggaagtatggctatggaatctgccaaggagactcgctactgtgcagtgtgcaatgactatgcttcaggctaccattatggagtctggtcctgtgagggctgcaaggccttcttcaagagaagtattcaaggacataacgactatatgtgtccagccaccaaccagtgcaccattgataaaaacaggaggaagagctgccaggcctgccggctccgcaaatgctacgaagtgggaatgatgaaaggtgggatacgaaaagaccgaagaggagggagaatgttgaaacacaagcgccagagagatgatggggagggcaggggtgaagtggggtctgctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacagggagctggttcacatgatcaactgggcgaagagggtgccaggctttgtggatttgaccctccatgatcaggtccaccttctagaatgtgcctggctagagatcctgatgattggtctcgtctggcgctccatggagcacccagggaagctactgtttgctcctaacttgctcttggacaggaaccagggaaaatgtgtagagggcatggtggagatcttcgacatgctgctggctacatcatctcggttccgcatgatgaatctgcagggagaggagtttgtgtgcctcaaatctattattttgcttaattctggagtgtacacatttctgtccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgagtaacaaaggcatggagcatctgtacagcatgaagtgcaagaacgtggtgcccctctatgacctgctgctggagatgctggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccacggtctga

SEQ ID NO: 2 - protein, 595 aa, Homo sapiens [sequence omitted]

SEQ ID NO: 3 - DNA, 882 bp, Homo sapiens
aagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacagggagctggttcacatgatcaactgggcgaagagggtgccaggctttgtggatttgaccctccatgatcaggtccaccttctagaatgtgcctggctagagatcctgatgattggtctcgtctggcgctccatggagcacccagggaagctactgtttgctcctaacttgctcttggacaggaaccagggaaaatgtgtagagggcatggtggagatcttcgacatgctgctggctacatcatctcggttccgcatgatgaatctgcagggagaggagtttgtgtgcctcaaatctattattttgcttaattctggagtgtacacatttctgtccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgagtaacaaaggcatggagcatctgtacagcatgaagtgcaagaacgtggtgcccctctatgacctgctgctggagatgctggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccacggtc

SEQ ID NO: 4 - protein, 294 aa, Homo sapiens
KKNSLALSLTADQMVSALLDAEPPILYSEYDPTRPFSEASMMGLLTNLADRELVHMINWAKRVPGFVDLTLHDQVHLLECAWLEILMIGLVWRSMEHPGKLLFAPNLLLDRNQGKCVEGMVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEHLYSMKCKNVVPLYDLLLEMLDAHRLHAPTSRGGASVEETDQSHLATAGSTSSHSLQKYYITGEAEGFPATV

SEQ ID NO: 5 - DNA, 1593 bp, Homo sapiens
atggatataaaaaactcaccatctagccttaattctccttcctcctacaactgcagtcaatccatcttacccctggagcacggctccatatacataccttcctcctatgtagacagccaccatgaatatccagccatgacattctatagccctgctgtgatgaattacagcattcccagcaatgtcactaacttggaaggtgggcctggtcggcagaccacaagcccaaatgtgttgtggccaacacctgggcacctttctcctttagtggtccatcgccagttatcacatctgtatgcggaacctcaaaagagtccctggtgtgaagcaagatcgctagaacacaccttacctgtaaacagagagacactgaaaaggaaggttagtgggaaccgttgcgccagccctgttactggtccaggttcaaagagggatgctcacttctgcgctgtctgcagcgattacgcatcgggatatcactatggagtctggtcgtgtgaaggatgtaaggccttttttaaaagaagcattcaaggacataatgattatatttgtccagctacaaatcagtgtacaatcgataaaaaccggcgcaagagctgccaggcctgccgacttcggaagtgttacgaagtgggaatggtgaagtgtggctcccggagagagagatgtgggtaccgccttgtgcggagacagagaagtgccgacgagcagctgcactgtgccggcaaggccaagagaagtggcggccacgcgccccgagtgcgggagctgctgctggacgccctgagccccgagcagctagtgctcaccctcctggaggctgagccgccccatgtgctgatcagccgccccagtgcgcccttcaccgaggcctccatgatgatgtccctgaccaagttggccgacaaggagttggtacacatgatcagctgggccaagaagattcccggctttgtggagctcagcctgttcgaccaagtgcggctcttggagagctgttggatggaggtgttaatgatggggctgatgtggcgctcaattgaccaccccggcaagctcatctttgctccagatcttgttctggacagggatgaggggaaatgcgtagaaggaattctggaaatctttgacatgctcctggcaactacttcaaggtttcgagagttaaaactccaacacaaagaatatctctgtgtcaaggccatgatcctgctcaattccagtatgtaccctctggtcacagcgacccaggatgctgacagcagccggaagctggctcacttgctgaacgccgtgaccgatgctttggtttgggtgattgccaagagcggcatctcctcccagcagcaatccatgcgcctggctaacctcctgatgctcctgtcccacgtcaggcatgcgagtaacaagggcatggaacatctgctcaacatgaagtgcaaaaatgtggtcccagtgtatgacctgctgctggagatgctgaatgcccacgtgcttcgcgggtgcaagtcctccatcacggggtccgagtgcagcccggcagaggacagtaaaagcaaagagggctcccagaacccacagtctcagtga

SEQ ID NO: 6 - protein, 530 aa, Homo sapiens
MDIKNSPSSLNSPSSYNCSQSILPLEHGSIYIPSSYVDSHHEYPAMTFYSPAVMNYSIPSNVTNLEGGPGRQTTSPNVLWPTPGHLSPLVVHRQLSHLYAEPQKSPWCEARSLEHTLPVNRETLKRKVSGNRCASPVTGPGSKRDAHFCAVCSDYASGYHYGVWSCEGCKAFFKRSIQGHNDYICPATNQCTIDKNRRKSCQACRLRKCYEVGMVKCGSRRERCGYRLVRRQRSADEQLHCAGKAKRSGGHAPRVRELLLDALSPEQLVLTLLEAEPPHVLISRPSAPFTEASMMMSLTKLADKELVHMISWAKKIPGFVELSLFDQVRLLESCWMEVLMMGLMWRSIDHPGKLIFAPDLVLDRDEGKCVEGILEIFDMLLATTSRFRELKLQHKEYLCVKAMILLNSSMYPLVTATQDADSSRKLAHLLNAVTDALVWVIAKSGISSQQQSMRLANLLMLLSHVRHASNKGMEHLLNMKCKNVVPVYDLLLEMLNAHVLRGCKSSITGSECSPAEDSKSKEGSQNPQSQ

SEQ ID NO: 7 - protein, 583 aa, Acanthopagrus schlegelii
MYPEDSRVSGGVATVDFLEGTYDYAAPTPAPTPLYSHSTPGYYSAPLDAHGPPSDGSLQSLGSGPNSPLVFVPSSPRLSPFMHPPTHHYLETTSTPIYRSSVPSSQHSASREDQCGTSDDSYSVGESGAGAGAAGFEMAKEMRFCAVCSDYASGYHYGVWSCEGCKAFFKRSIQGHNDYMCPATNQCTIDRNRRKSCQACRLRKCYEVGMMKGGVRKDRGRVLRRDKRRTGTSDRDKASKGLEHRTAPPQDRRKHISSSAAGGGGKSSVISMPPDQVLLLLQGAEPPMLCSRQKVNRPYTEVTVMTLLTSMADKELVHMIAWAKKLPGFLQLSLHDQVQLLESSWLEVLMIGLIWRSIHCPGKFIFAQDFILDRSEGDCVEGMAEIFDMLLATASRFRMLKLKPEEFVCLKAIVLLNSGAFSFCTGTMEPLHDGAAVQNMLDTITDALIHHINQSGCTAQQQSRRQAQLLLLLSHIRHMSNKGMEHLYSMKCKNKVPLYDLLLEMLDAHRVHRPDRPHETWSQADREPPFTSRNNRGSGGGGGSSSAGSTSGTRVSLENPTGPGVLQYGRSAPSAPHPMKPTE

SEQ ID NO: 8 - protein, 587 aa, Alligator mississippiensis
MTMTLHTKTSGVTLLHQIQGTELETLSRPQLKIPLDRSLSEMYVESNKTGIFNYPEGTTYDFATAAPVYSSTSLSYAPTSESYGSSSLGGFHSLNNVPPSPVVFLQTAPQLSPFIHHHSQQVPYYLENDQSGFGMREAAPSTFYRPGADSRRQSGRERMSSTSEKTSLSMESTKETRYCAVCNDYASGYHYGVWSCEGCKAFFKRSIQGHNDYMCPATNQCTIDKNRRKSCQACRLRKCYEVGMMKGGIRKDRRGGRMLKQKRQREEQDARNGETATAEMRTPTLWTSPLVIKHTKKNSPALSLTAEQMVSALLEAEPPIVYSEYDPNRPFNEASMMTLLTNLADRELVHMINWAKRVPGFVDLTLHDQVHLLECAWLEILMIGLVWRSVEHPGKLLFAPNLLLDRNQGKCVEGMVEIFDMLLATAARFRMMNLQGEEFVCLKSIILLNSGVYTFLSSTLKSLEEKDYIHRVLDKITDTLIHLMAKSGLSLQQQHRRLAQLLLILSHIRHMSNKGMEHLYNMKCKNVVPLYDLLLEMLDAHRLHAPAARNAAQVEEETRLTTASASSHSLQSFYINNREDENLQNTI

SEQ ID NO: 9 - protein, 582 aa, Astatotilapia burtoni
MYPEESRGSGGVATVDFLEGSYDYAAPTPAPTPLYSHSTTGCYSAPLDAHGPPSDGSLQSLGSGTTSPLVFVPSSPRLSPFMHPPSHHYLETTSTPVYRSSHQPVPRDDQCGTRDEAYGLGELGAGAGGFEMTKETRFCAVCSDYASGYHYGVWSCEGCKAFFKRSIQGHNDYMCPATNQCTIDKNRRKSCQACRLRKCYEVGMMKGGMRKDRGRVLRREKRRAYDRDKPAKDLPHTKAPPHDGRKHATSSSSTSGGGGRSSLNSIPPDQVLLLLQGAEPPTLCSRQKMNQPYTEVTMMTLLTSMADKELVHMIAWAKKLPGFLQLSLHDQVLLLESSWLEVLMIGLIWRSIHCPGKLIFAQDLILDRTEGTCVEGMAEIFDMLLATASRFRMLKLKPEEFVCLKAIILLNSGAFSFCTGTMEPLHDSAAVQHMLDTITDALIFHISQLGCSAQHQSRRQAQLLLLLSHIRHMSNKGMEHLYSMKCKNKVPLYDLLLEMLDAQRIHRPVKPSQSWSQGDRDSPNTSSSGGGGSDDEGTSSAGSSSGPQGNHESPRCENLSRAPTGPGVLQYRGSHSDCTPIL

SEQ ID NO: 10 - protein, 596 aa, Bos taurus
MTMTLHTKASGMALLHQIQANELEPLNRPQLKIPLERPLGEVYMDSSKPAVYNYPEGAAYDFNAAAPASAPVYGQSGLPYGPGSEAAAFGANGLGAFPPLNSVSPSPLVLLHPPPQPLSPFLHPHGQQVPYYLENESSGYAVREAGPPAYYRPNSDNRRQGGRERLASTSDKGSMAMESAKETRYCAVCNDYASGYHYGVWSCEGCKAFFKRSIQGHNDYMCPATNQCTIDKNRRKSCQACRLRKCYEVGMMKGGIRKDRRGGRMLKHKRQRDDGEGRNEAVPSGDMRAANLWPSPIMIKHTKKNSPVLSLTADQMISALLEAEPPIIYSEYDPTRPFSEASMMGLLTNLADRELVHMINWAKRVPGFVDLALHDQVHLLECAWLEILMIGLVWRSMEHPGKLLFAPNLLLDRNQGKCVEGMVEIFDMLLATSSRFRMMNLQGEEFVCLKSIILLNSGVYTFLSSTLRSLEEKDHIHRVLDKITDTLIHLMAKAGLTLQQQHRRLAQLLLILSHFRHMSNKGMEHLYSMKCKNVVPLYDLLLEMLDAHRLHAPANFGSAPPEDVNQSQLAPTGCTSSHSLQTYYITGEAENFPSTV

SEQ ID NO: 11 - protein, 587 aa, Caiman crocodilus
MTMTLHTKTSGVTLLHQIQGTELETLSRPQLKIPLDRSLSEMYVENNKTGIFNYPEGTTYDFATAAPVYSSTSLSYAPTSESYGSSSLGGFHSLNNVPPSPVVFLQTAPQLSPFVHHHSQQVPYYLENDQSGFGMREAASSTFYRPSADSRHQSGRERMSSTSEKASLSMESTKETRYCAVCNDYASGYHYGVWSCEGCKAFFKRSIQGHNDYMCPATNQCTIDKNRRKSCQACRLRKCYEVGMMKGGIRKDRRGGRMLKQKRQREEQDARNGETATAEMRTPTLWTSPLVIKHTKKNSPALSLTAEQMVSALLEAEPPIVYSEYDPNRPFNEASMMTLLTNLADRELVHMINWAKRVPGFVDLTLHDQVHLLECAWLEILMIGLVWRSMEHPGKLLFAPNLLLDRNQGKCVEGMVEIFDMLLATAARFRMMNLQGEEFVCLKSIILLNSGVYTFLSSTLKSLEEKDYIHRVLDKITDTLIHLMAKSGLSLQQQHRRLAQLLLILSHIRHMSNKGMEHLYNMKCKNVVPLYDLLLEMLDAHRLHAPAARNAAQVEEETRLTTASASSHSLQSFYINNREDENLQNTI

SEQ ID NO: 12 - protein, 353 aa, Cavia porcellus
RKCYDVGMIKGGIRKDRRGGRMLKYKRQRDDEERRNEMGPSGDMRGSNLWPSPLVIKHTKKNSPALSLTADQMVSALMDAEPPLLYSEYDAVKPFSEASMMGLLTNLADRELVHMINWAKRVPGFGDLTLHDQVHLLECAWLEILMIGLIWRSMEHPGKLLFAPNLILDRNQGKCVEGMVEIFDMLLATSTRFRMMNLQGEEFVCLKSIILLNSGMYTFLSSTLKSLEEKDHIHRVLDKIIDTLIHLMAKAGLTLQQQHRRLAQLLLILSHIRHMSNKGVEHLYNMKCKNVVPLYNLLLEMLEAHRLNTSSNPMGGSPEEPSQSQLATIGSSSAHSLQTYYISQEAESFPNTI

SEQ ID NO: 13 - protein, 581 aa, Chrysophrys major
MYPEDSRGSGGVATVDFLEGTYDYAAPTPAPTPLYSHSTPGYYSAPLDAHGPPSDGSLQSLGSGPNSPLVFVPSSPRLSPFMHPPTHHYLETTSTPVYRSSVPSSQQSVSREDQCGTSDDSYSVGESGAGALAAGFEIAKEMRFCAVCSDYASGYHYGVWSCEGCKAFFKRSIQGHNDYMCPATNQCTIDRNRRKSCQACRLRKCYEVGMMKGGMRKDRGRVLRRDKQRTGTSDRDKASKGLEHRTAPPQDRRKHISSSAGGGGGKSSMISMPPDQVLLLLQGAEPPMLCSRQKLNRPYTEVTMMTLLTSMADKELVHMIAWAKKLPGFLQLSLHDQVQLLESSWLEVLMIGLIWRSIHCPGKLIFAQDLILDRSEGDCVEGMAEIFDMLLATASRFRMLKLKPEEFVCLKAIILLNSGAFSFCTGTMEPLHDGAAVQNMLDTITDALIHHINQSGCSAQQQSRRQAQLLLLLSHIRHMSNKGMEHLYSMKCKNKVPLYDLLLEMLDAHRIHRADRPAETWSQADREPPFTSRNSSGGGGGGGGGSSSAGSTSGPRVSHESPTSPGVLQYGGSRSECTHIL

SEQ ID NO: 14 - protein, 589 aa, Coturnix japonica
MTMTLHTKASGVTLLHQIQGTELETLSRPQLKIPLERSLSDMYVESNKTGVFNYPEGATYDFGTTAPVYGSTTLSYAPTSESFGSSSLAGFHSLNNVPPSPVVFLQTAPQLSPFIHHHSQQVPYYLENEQGSFGMRETAPPAFYRPSSDNRRHSIRERMSSASEKGSLSMESTKETRYCAVCNDYASGYHYGVWSCEGCKAFFKRSIQGHNDYMCPATNQCTIDKNRRKSCQACRLRKCYEVGMMKGGIRKDRRGGRMMKQKRQREEQESRNGEASSTELRAPTLWTSPLVVKHNKKNSPALSLTAEQMVSALLEAEPPIVYSEYDPNRPFNEASMMTLLTNLADRELVHMINWAKRVPGFVDLTLHDQVHLLECAWLEILMIGLVWRSMEHPGKLLFAPNLLLDRNQGKCVEGMVEIFDMLLATAARFRMMNLQGEEFVCLKSIILLNSGVYTFLSSTLKSLEERDYIHRVLDKITDTLIHFMAKSGLSLQQQHRRLAQLLLILSHIRHMSNKGMEHLYNMKCKNVVPLYDLLLEMLDAHRLHAPAARSAAPMEEENRSQLTTAPASSHSLQSFYINSKEEESMQNTI

SEQ ID NO: 15 - protein, 569 aa, Danio rerio
MYPKEEHSAGGISSSVNYLDGAYEYPNPTQTFGTSSPAEPASVGYYPAPPDPHEEHLQTLGGGSSSPLMFAPSSPQLSPYLSHHGGHHTTPHQVSYYLDSSSSTVYRSSVVSSQQAAVGLCEELCSATDRQELYTGSRAAGGFDSGKETRFCAVCSDYASGYHYGVWSCEGCKAFFKRSIQGHNDYVCPATNQCTIDRNRRKSCQACRLRKCYEVGMMKGGIRKDRGGRSVRRERRRSSNEDRDKSSSDQCSRAGVRTTGPQDKRKKRSGGVVSTLCMSPDQVLLLLLGAEPPAVCSRQKHSRPYTEITMMSLLTNMADKELVHMIAWAKKVPGFQDLSLHDQVQLLESSWLEVLMIGLIWRSIHSPGKLIFAQDLILDRSEGECVEGMAEIFDMLLATVARFRSLKLKLEEFVCLKAIILINSGAFSFCSSPVEPLMDNFMVQCMLDNITDALIYCISKSGASLQLQSRRQAQLLLLLSHIRHMSNKGMEHLYRMKCKNRVPLYDLLLEMLDAQRFQSSGKVQRVWSQSEKNPPSTPTTSSSSSNNSPRGGAAAIQSNGACHSHSPDP

SEQ ID NO: 16 - protein, 594 aa, Equus caballus
MTMTLHTKASGMALLHQIQGNELETLNLPQFKIPLERPLGEVYVESSKPPVYDYPEGAAYDFNAAAAASASVYGQSGLAYGPGSEAAAFGANGLGGFPPLNSVSPSQLMLLHPPPQLSPYLHPPGQQVPYYLENEPSGYSVCEAGPQAFYRPNADNRRQGGRERLASSGDKGSMAMESAKETRYCAVCNDYASGYHYGVWSCEGCKAFFKRSIQGHNDYMCPATNQCTIDKNRRKSCQACRLRKCYEVGMMKGGIRKDRRGGRMLKHKRQRDDGEGRNEAGPSGDRRPANFWPSPLLIKHTKKISPVLSLTAEQMISALLDAEPPVLYSEYDATRPFNEASMMGLLTNLADRELVHMINWAKRVPGFVDLSLHDQVHLLECAWLEILMIGLVWRSMEHPGKLLFAPNLLLDRNQGKCVEGMVEIFDMLLATSSRLRMMNLQGEEFVCLKSIILLNSGVYTFLSSTLKSLEEKDHIHRVLDKMTDTLIHLMAKAGLTLQQHRRLAQLLLILSHIRHMSNKGMEHLYSMKCKNVVPLYDLLLEMLDAHRLHAPANHGGAPMEETNQSQLATTGSTSPHSMQTYYITGEAEGFPNTI

SEQ ID NO: 17 - protein, 573 aa, Fundulus heteroclitus
MYPEESRGSGGVAAVDFLEGTYDYATPTPAPTPLYSHSTTGYYSAPLDAQGPPSDGSLHSLGSGPTSPLVFVPTSPRLSLFMHAPSQHYLETASTPVYRSSHQPASREDQCDTRDEACSVGELGAGAGAGAAAGGFEMAKETRFCAVCSDYASGYHYGVWSCEGCKAFFKRSIQGHNDYMCPATNQCTIDRNRRKSCQACRLRKCYEVGMMKGGVRKERGRVLRRDKRRTAISDREKAVKGLEPKTSPHQDKRRRGSALGGDRSSVASLPSEQVLLLFQGAEPPILCSRQKLSRPYTEVTMMTLLTSMADKELVHMIAWAKKLPGFLQLALHDQVLLLESSWLEVLMIGLIWRSIHCPGKLIFAQDLILDRNEGDCVEGMTEIFDMLLATASRFRMLKLKPEEFVCLKAIILLNSGAFSFCTGTMEPLHDSVAVQNMLDTITDALIHHISQSGFSVQQQARRQAQLLLLLSHIRHMSNKGMEHLYSMKCKNKVPLYDLLLEMLDAHRHHPVKPSQDGKSPPSTSSFGAGCEGGSSSAGSSSGPRGSGDNLMRIHSAPGVLQYGGSRSDCAQVL

SEQ ID NO: 18 - protein, 574 aa, Halichoeres tenuispinis
MYPEESRGSGGVGTVDFLEGTYDYTAPTPAPTLYSLSTQGYYSAALDTHGQPSDSSIQSLGSGPSSPLVFVPSSPRLSPFMHLPSHHYLETSSTPVYRSSVSSSQQSISREEHCGTSDESYSMGESGAGAAAGCFEMAKEMRYCAVCSDYASGYHYGVWSCEGCKAFFKRSIQGHNDYMCPATNQCTIDRNRRKSCQACRLRKCYEVGMMKGGVRKDRGRVLRRDKRRTGTSDKDNGSKDREQRTVPPQGRRKHGSSVGGGKSPVISMPPDQVLLLLQGAEPPILCSRQKLSRPYTEVTMMTLLTSMTDRELVHMIAWAKKLPGFLQLTLHDQVQLLESSWLEVLMIGLIWRSIHCPGKLIFAQDLILDRSEGDCVEGMAEIFDMLLATTSRFRMLKLKPEEFVCLKAIILLNSGAFSFCTGTMEPLHDNEAVQNMLDIITDALIHHISQSGCSAHQQSRRQAQLLLLLSHIRHMSNKGMEHLYSMKCKNKVPLYDLLLEMLDAHRLHRPDRPAESWYQTDREPAYSSSATTTNDNSSSSPAGSRASQESPNRPPTGHSVLQFGGSRSDCTHIL

SEQ ID NO: 19 - protein, 458 aa, Halichoeres trimaculatus
SDESYGMGESGAGAAAGCFEMAKEMRYCAVCSDYASGYHYGVWSCEGCKAFFKRSIQGHNDYMCPATNQCTIDRNRRKSYQACRLRKCYEVGMMKGGARKDRGRVLRRDKRRTCTSDKDKGSKERDERTAPPQAGGNTATVWEENPQWISMPPDQVLLLLQGAETPILYSRQKLSRPYTEVTMMTLLTSMADRELVHMIAWAKKLPGFLQLTHHDQVQLLESSWLEVLMIGLIWRSIHCRGKHIFAQDLILDRNEGDCVEGMAEIFDMLLATTSPFRMLKLKPEEFVCLKAIVLLNSGAFSFCTGTMEPLHDSAPVQDMLDIITDALIHHISQSGCSAHQQSRRQAQLLLLLSHIRHMSNKGMEHLYSMKCKNKVPLYDLLLEMLDAHRLHRPDRPAESWSQTDGEPAYSSSATTTNDSNNNSSSAGSRAGHEGPNKPPTSPGVLQYGGSRSDCTHVL

SEQ ID NO: 20 - protein, 581 aa, Ictalurus punctatus
MYPEEEQRTTGGISSTAHYLDGTFNYTTNPDATNSSVDYYSVAPEPQEENLQPLPNGSSSPPVFVPSSPQLSPFLGHPPAGQHTAQQVPYYLEPSGTSIYRSSVLASAGSRVELCSAPGRQDVYTAVGASGPSGASGPSGAIGLVKEIRYCSVCSDYASGYHYGVWSCEGCKAFFKRSIQGHNDYVCPATNQCTIDRNRRKSCQACRLRKCYEVGMMKGGFRKERGGRIIKHNRRPSGLKERERGYSKAQSGSDVREALPQDGQSSSGIGGGVADVVCMSPEQVLLLLLRAEPPTLCSRQKHSRPYSELTIMSLLTNMADRELVHMIAWAKKVPGFQDLSLHDQVQLLESSWLEILMIGLIWRSIYTPGKLIFAQDLILDKSEGECVEGMAEIFDMLLATVARFRTLKLKSEEFVCLKAIILLNSGAFSFCSSPVEPLRDGFMVQCMMDNITDALIYYISQSGISVQLQSRRQAQLLLLLSHIRHMSYKGMEHLYSMKCKNKVPLYDLLLEMLDAHRLRPLGKVPRIWADRVSSSPTTTATTPTTNTTTTTTTTTHHPSNGSTCPADLPSNPPGPGQSPSP

SEQ ID NO: 21 - protein, 627 aa, Micropterus salmoides
MCKRQSPAQSKQPCGTVLRPRIGPAFTELETLSPQHPSPPLRAPLSDMYPEESRGSGGGATVDFLEGTYDYVAPTPVPTPLYSHSGYYSAPLDAQGPPSDGSLQSLGSGPTSPLVFVPSSPRLSPFMHPPSHHYLETTSTPVYRSSVLSSQQPVPREDQCATSDESYCVGESGAGAGGFEMAKEMRFCAVCSDYASGYHYGVWSCEGCKAFFKRSIQGHNDYMCPATNQCTIDRNRRKSCQACRLRKCYEVGMMKGGVRKDRGRVLRRDKRRAGTNDRDKASKDLEYRTVPPQDRRKHSSSSAGGGGGKSSVTGMSPDQVLLLLQGAEPPMLCSRQKLSRPYTEVTIMTLLTSMADKELVHMITWAKKLPGFLQLSLHDQVQLLESSWLEVLMIGLIWRSIHCPGKLIFAQDLILDRNEGDCVEGFVEIFDMLLATASRFRMLKLKPEEFVCLKAIILLNSGAFSFCTGTMEPLHNSVEVHNMLDTITDALIHHISQSGCSAQQQSRRQAQLLLLLSHIRHMSNKGMEHLYSMKCKNKVPLYDLLLEMLDAHRIHRPDRPAQFWSQADGEPPFITVNNCNSSSNGGVSSSVGSSSGPRVSHESPSRGPTGPGVLQYGGSRSDCTHIL

SEQ ID NO: 22 - protein, 599 aa, Mus musculus
MTMTLHTKASGMALLHQIQGNELEPLNRPQLKMPMERALGEVYVDNSKPTVFNYPEGAAYEFNAAAAAAAAASAPVYGQSGIAYGPGSEAAAFSANSLGAFPQLNSVSPSPLMLLHPPPQLSPFLHPHGQQVPYYLENEPSAYAVRDTGPPAFYRSNSDNRRQNGRERLSSSNEKGNMIMESAKETRYCAVCNDYASGYHYGVWSCEGCKAFFKRSIQGHNDYMCPATNQCTIDKNRRKSCQACRLRKCYEVGMMKGGIRKDRRGGRMLKHKRQRDDLEGRNEMGASGDMRAANLWPSPLVIKHTKKNSPALSLTADQMVSALLDAEPPMIYSEYDPSRPFSEASMMGLLTNLADRELVHMINWAKRVPGFGDLNLHDQVHLLECAWLEILMIGLVWRSMEHPGKLLFAPNLLLDRNQGKCVEGMVEIFDMLLATSSRFRMMNLQGEEFVCLKSIILLNSGVYTFLSSTLKSLEEKDHIHRVLDKITDTLIHLMAKAGLTLQQQHRRLAQLLLILSHIRHMSNKGMEHLYNMKCKNVVPLYDLLLEMLDAHRLHAPASRMGVPPEEPSQTQLATTSSTSAHSLQTYYIPPEAEGFPNTI

SEQ ID NO: 23 - protein, 643 aa, Ovis aries
SLPSHCLSPLLQAHGTFLERRSSSRVAGRLLSPLPRGETVCAGPRLTMTMTLHTKASGMALLHQIQANELEPLNRPQLKIPLERPLGEMYVDSSKPAVYNYPEGAAYDFNAAAAASAPVYGQSGLPYGPGSEAAAFGANGLGAFPPLNSVSPSPLVLLHPPPQPLSPFLHPHGQQVPYYLENEPSGYAVREAGPPAYYRPNSDNRRQGGRERLASTSDKGSMAMESAKETRYCAVCNDYASGYHYGVWSCEGCKAFFKRSIQGHNDYMCPATNQCTIDKNRRKSCQACRLRKCYEVGMMKGGIRKDRRGGRMLKHKRQRDDGEGRNEAVPSGDMRATNLWPSPIMIKHTKKNSPVLSLTADQMISALLEAEPPIIYSEYDPTRPFSEASMMGLLTGLADRELVHMINWAKRVPGFVDLALHDQVHLLECAWLEILMIGLVWRSMEHPGKLLFAPNLLLDRNQGKCVEGMVEIFDMLLATSSRFRMMNLQGEEFVCLKSIILLNSGVYTFLSSTLRSLEEKDHIHRVLDKITDTLIHLMAKAGLTLQQQHRRLAQFLLLLSHFRHMSNKGMEHLYSMKCKNVVPLYDLLLEMLDAHRLHAPANFGSTPPEDVNQSQLATTGCTSSHSLQTYYITGEAENFPSTV

SEQ ID NO: 24 - protein, 620 aa, Oncorhynchus masou
MLVRQSHTQISKPLGAPLRSRTTLESHVISPTKLSPQQPTTPNSNMYPEETRGGGGAAAFNYLDGGYDYTAPAQGPAPLYYSTTPQDAHGPPSDGSMQSLGSSPTGPLVFVSSSPQLSPQLSPFLHPPSHHGLPSQSYYLETSSTPLYRSSVVTNQLSASEEKLCIASDRQQSYSAAGSGVRVFEMANETRYCAVCSDFASGYHYGVWSCEGCKAFFKRSIQGHNDYMCPATNQCTMDRNRRKSCQACRLRKCYEVGMVKGGLRKDRGGRVLRKDKRYCGPAGDREKPYGDLEHRTAPPQDGVRNSSSSLNGGGGWRGPRITMPPEQVLFLLQGAEPPALCSRQKVARPYTEVTMMTLLTSMADKELVHMIAWAKKVPGFQELSLHDQVQLLESSWLEVLMIGLIWRSIHCPGKLIFAQDLILDRSEGDCVEGMAEIFDMLLATVSRFRMLKLKPEEFLCLKAIILLNSGAFSFCSNSVESLHNSSAVESMLDNITDALIHHISHSGASVQQQPRRQAQLLLLLSHIRHMSNKGMEHLYSIKCKNKVPLYDLLLEMLDGHRLQSPGKVAQAGEQTEGPSTTTTTSTGSSIGPMRGSQDTHIRSPGVLQYGSPSSDQMPIP

SEQ ID NO: 25 - protein, 578 aa, Paralichthys olivaceus
MYPEESRGSGGAATVDFLEGTYDYAAPTPAQTPLYSHSTSGYYSAPLDAHGPPSDGSRHSLGSGPTSPHVYVPSSPRLSPFMHPPSHHYLETTATSVYRSSQQPVTREDHCGPRDESFSVGETGAAAGAEGFEMAKETRFCAVCSDYASGYHYGVWSCEGCKAFFKRSIQGHNDYMCPATNQCTIDRNRRKSCQACRLRKCYEVGMMKGGVRKDRSHVLRRDKRRAGTNDRDKASKDQDHKTVPLQDGRKSSSSTAGGKSSVTAMLPDQVLVLLQGAEPPILCSRQKLNQPYTEVTMMTLLTSMADRELVHMIAWAKKLPGFLQLSLHDQVQLLESSWLEVLMIGLIWRSIHCPGKLIFAQDLILDRNEGNCVEGMAEIFDMLLATASRFRMLKLKSEEFFCLKAIILLNSGSFSFCTGTMEPLHNTAAVQDMLETITDALIHHISQSGCPVQQQWRRQAQLLLLLSHIRHMSNKGMEHLYSMKCKNKVPLYDLLLEMLDAHCLHRPARPAQSWLQADREPSAAGNNNNNSSSIIISGGGSSSASSGHRGSQESPSRATTGPSVLQHGGSRPDCTHIL

SEQ ID NO: 26 - protein, 579 aa, Sparus aurata
MYPEDSRVSGGVATVDFLEGTYDYAAPTPAPTPLYSHSTPGYYSAPLDAHGPPSDGSLQSLGSGPNSPLVFVPSSPHLSPFMQPANHHYLETTSTPIYSVPSSQHSVSREDQCGTSDDSYSVGESGAGAGAAGFEMAKEMRFCAVCSDYASGYHYGVWSCEGCKAFFKRSIQGHNDYMCPATNQCTIDRNRRKSCQACRLRKCYEVGMMKGGVRKDRGRVLRRDKRRTGTSDRDKASKGLEHRTAPPQDRRKHISSSAGGGGGKSSVISMPPDQVLLLLRGAEPPMLCSRQKVNRPYTEVTVMTLLTSMADKELVHMIAWAKKLPGFLQLSLHDQVQLLESSWLEVLMIGLIWRSIHCPGKLIFAQDLILDRSEGDCVEGMAEIFDMLLATASRFRMLKLKPEEFVCLKAIILLNSGAFSFCTGTMEPLHDSAAVQNMLDTITDALIHHINQSGCSAQQQSRRQAQLLLLLSHIRHMSNKGMEHLYSMKCKNKVPLYDLLLEMLDAHRVHRPDRPAETWSQADREPLFTSRNSSSSSGGGGGGSSSAGSTSGPQVNLESPTGPGVLQLRVHPHPMKPTE

SEQ ID NO: 27 - protein, 587 aa, Taeniopygia guttata
MTLHTKTSGVTLLHQIQGTELETLSRPQLKIPLERSLSDMYVETNKTGVFNYPEGATYDFGTTAPVYSSTTLSYAPTSESFGSSSLAGFHSLNSVPPSPVVFLQTAPHWSPFIHHHSQQVPYYLENDQGSFGMREAAPPAFYRPNSDNRRHSIRERMSSANEKGSLSMESTKETRYCAVCNDYASGYHYGVWSCEGCKAFFKRSIQGHNDYMCPATNQCTIDKNRRKSCQACRLRKCYEVGMMKGGIRKDRRGGRVMKQKRQREEQDSRNGEASSTELRAPTLWASPLVVKHNKKNSPALSLTAEQMVSALLEAEPPLVYSEYDPNRPFNEASMMTLLTNLADRELVHMINWAKRVPGFVDLTLHDQVHLLECAWLEILMIGLVWRSMEHPGKLLFAPNLLLDRNQGKCVEGMVEIFDMLLATAARFRMMNLQGEEFVCLKSIILLNSGVYTFLSSTLKSLEEKDYIHRVLDKITDTLIHLMAKSGLSLQQQHRRLAQLLLILSHIRHMSNKGMEHLYNMKCKNVVPLYDLLLEMLDAHRLHAPAARSAAPMEEENRSQLTTASASSHSLQSFYINSKEEENMQNTL

SEQ ID NO: 28 - protein, 585 aa, Tilapia nilotica
MYPEESRGSGGVATVDFLEGTYDYAAPTPAPTPLYSHSTTGCYSAPLDAHGPLSDGSLQSLGSGPTSPLVFVPSSPRLSPFMHPPSHHYLETTSTPVYRSSHQPVPREDQCGTRDEAYSVGELGAGAGGFEMTKDTRFCAVCSDYASGYHYGVWSCEGCKAFFKRSIQGHNDYMCPATNQCTIDKNRRKSCQACRLRKCYEVGMMKGGMRKDRGRVLRREKRRACDRDKPAKDLPHTRASPQDGRKRAMSSSSTSGGGGRSSLNNMPPDQVLLLLQGAEPPILSSRQKMSRPYTEVTIMTLLTSMADKELVHMITWAKKLPGFLQLSLHDQVLLLESSWLEVLMIGLIWRSIQCPGKLIFAQDLILDRNEGTCVEGMAEIFDMLLATASRFRVLKLKPEEFVCLKAIILLNSGAFSFCTGTMEPLHDSAAVQHMLDTITDALIFHISHLGCSAQQQSRRQAQLLLLLSHIRHMSNKGMEHLYSMKCKNKVPLYDLLLEMLDAHRIHRPVKPFQSWSQGDRDSPTASSTSSSGGGGGDDEGASSAGSSSGPQGSHESPRRENLSRAPTGPGVLQYRGSHSDCTRIP

SEQ ID NO: 29 - protein, 427 aa, Xenopus laevis
MSSANDKGPPSMESTKETRFCAVCSDYASGYHYGVWSCEGCKAFFKRSIQGHNDYMCPATNQCTIDKNRRKSCQACRLRKCYEVGMMKGGIRKDRRGGRMLKHKQQKEEPEQKNDVNPSEIRTASIWVNPSVKSMKLSPVLSLTAEQLISALMEAEPPIVYSEHDSTKPLSEASMMTLLTNLADKELVHMINWAKRVPGFVDLTLHDQVHLLECAWLEILMVGLIWRSVEHPEKLSFAPNLLLDRNQGRCVEGLVEIFDMLVTTATRFRMMRLHGEEFICLKSIILLNSGVYTFLSSTLESLEDTDLIHIILDKIIDTLVHFMAKSGLSLQQQQRRLAQLLLILSHIRHMSNKGMEHLYSMKCKNVVPLYDLLLEMLDAHRIHTPKDKTTTQEEESRSPLSTTVNGASPCLQPFYKNTEEVSLQSTV

SEQ ID NO: 30 - protein, 58 aa, Artificial Sequence
  Mutant human estrogen receptor alpha C-terminus
LPCKSITSRGRQRVSLPQSEVDSRGSIRPGLEPGSTLEPYSESYYCSQANSGRISYDL

SEQ ID NO: 31 - DNA, 20 bp, Artificial Sequence
  Synthetic primer
cgacatcatcatcggaagag

SEQ ID NO: 32 - DNA, 20 bp, Artificial Sequence
  Synthetic primer
gcttggctgcagtaatacga